(19) United States
(12) Patent Application Publication
(10) Pub. No.: US 2021/0343383 A1
(43) Pub. Date: Nov. 4, 2021

(54) CUSTOMIZABLE COMMUNICATION PLATFORM WITH ALERT TAGS

(71) Applicant: CAREMINDR Corporation, Los Gatos, CA (US)

(72) Inventor: Harry Raymond Soza, San Jose, CA (US)

(21) Appl. No.: 17/378,655

(22) Filed: Jul. 16, 2021

Related U.S. Application Data
(63) Continuation-in-part of application No. 16/521,822, filed on Jul. 25, 2019.
(60) Provisional application No. 62/712,557, filed on Jul. 31, 2018.

(51) Int. Cl. G16H 20/00; G16H 10/20; G16H 80/00

(52) U.S. Cl. CPC G16H 20/00; G16H 80/00; G16H 10/20

(57) ABSTRACT

Processing patient information and other treatment plan information may require access to a patient profile and other third parties involved in the patient treatment plan. One example method includes linking a mobile device and a health care provider server, requesting a patient response to a message including a query of a health-related issue, wherein the response is at least one of biometric, objective and subjective, receiving the patient response to the query, determining based on at least one predetermined alert criteria an urgency level of the patient response based on the health-related issue, tagging the patient response as an urgent tagged response if the determined urgency level exceeds a predetermined urgency threshold of the patient for the health-related issue based on the at least one predetermined alert criteria and providing the request and the urgent tagged response to a healthcare provider.

### BACKGROUND

Conventionally, the approach to providing users with ongoing communications regarding a plan or other repetitive course of action may leave the majority of the work to the user. The smartphone and other personal computing devices are everywhere and are not being properly utilized when offering users with options for maintaining a course of treatment or a set of goals. The lack of action taken by the professional service provider and/or the user can lead to personal health problems and lost revenue for providers, insurers, etc., as well as the users.

### SUMMARY

Example embodiments of the present application provide at least a first example method of the present application includes at least one of linking a mobile device and a health care provider server, requesting a patient response to a message including a query of a health-related issue, wherein the response is at least one of biometric, objective and subjective, receiving the patient response to the query, determining based on at least one predetermined alert criteria an urgency level of the patient response based on the health-related issue, tagging the patient response as an urgent tagged response if the determined urgency level exceeds a predetermined urgency threshold of the patient for the health-related issue based on the at least one predetermined alert criteria and providing the request and the urgent tagged response to a healthcare provider.

A second example embodiment of the present application provide at least a non-transitory computer readable medium comprising instructions that, when read by a processor, cause the processor to perform at least one of linking a mobile device and a health care provider server, requesting from a patient pertaining to a health-related issue a response to a query, wherein the response is at least one of biometric, objective and subjective, receiving the response to the query, determining based on at least one predetermined alert criteria an urgency level of the response based on the patient health-related issue, tagging the response as an urgent tagged response if the determined urgency level exceeds a predetermined urgency threshold of the patient for the health-related issue based on the at least one predetermined alert criteria and providing the request and the urgent tagged response a healthcare provider.

A further example embodiment of the present application provides at least a system, comprising at least one cloud-based processor, and at least one memory electrically coupled to the at least one processor and storing an application, the processor performing at least one operation to link a mobile device and a health care provider server, request from a patient pertaining to a health-related issue a response to a query, wherein the response is at least one of biometric, objective and subjective, receive the response to the query, determine based on at least one predetermined alert criteria an urgency level of the patient response based on the health-related issue, tag the response as an urgent tagged response if the determined urgency level exceeds a predetermined urgency threshold of the patient for the health-related issue based on the at least one predetermined alert criteria and provide the request and the urgent tagged response a healthcare provider.

### BRIEF DESCRIPTION OF THE DRAWINGS

FIG. 1 illustrates an example of the integrated application platform according to example embodiments.

FIG. 2 illustrates a network configuration of the third party participants of the integrated application according to example embodiments.

FIG. 3 illustrates a user smartphone interface of an example treatment plan according to example embodiments.

FIG. 4A illustrates an example setup configuration for a new course of treatment according to example embodiments.

FIG. 4B illustrates an example database entry for the new course of treatment according to example embodiments.

FIG. 4C illustrates a flow diagram configuration for the new course of treatment according to example embodiments.

FIG. 4D illustrates an example list of messages for the ongoing course of treatment according to example embodiments.

FIG. 4E illustrates an example setup configuration for various courses of treatment according to example embodiments.

FIG. 4F illustrates an example set of details of an ongoing course of treatment according to example embodiments.

FIG. 4G illustrates an example network configuration of the various third parties involved in the application operation and compliance according to example embodiments.

FIG. 5 illustrates a logic module configured to process the input and output parameters of the application according to example embodiments.

FIG. 6 illustrates an example network entity device configured to store instructions, software, and corresponding hardware for executing the same, according to example embodiments of the present application.

FIG. 7A illustrates a first portion of a data file defining tags and levels of urgency, according to example embodiments of the present application.

FIG. 7B illustrates a second portion of the data file defining tags and levels of urgency, according to example embodiments of the present application.

FIG. 8 illustrates a patient reply, according to example embodiments of the present application.

FIG. 9A illustrates a first portion of an electronic report to a healthcare provider with alert tags, according to example embodiments of the present application.

FIG. 9B illustrates a second portion of the electronic report to a healthcare provider with alert tags, according to example embodiments of the present application.

FIG. 10 illustrates system architecture, according to example embodiments of the present application.

FIG. 11 illustrates a first method, according to example embodiments of the present application.

FIG. 12 illustrates a second method, according to example embodiments of the present application.

FIG. 13 illustrates a first non-transitory computer readable medium, according to example embodiments of the present application.

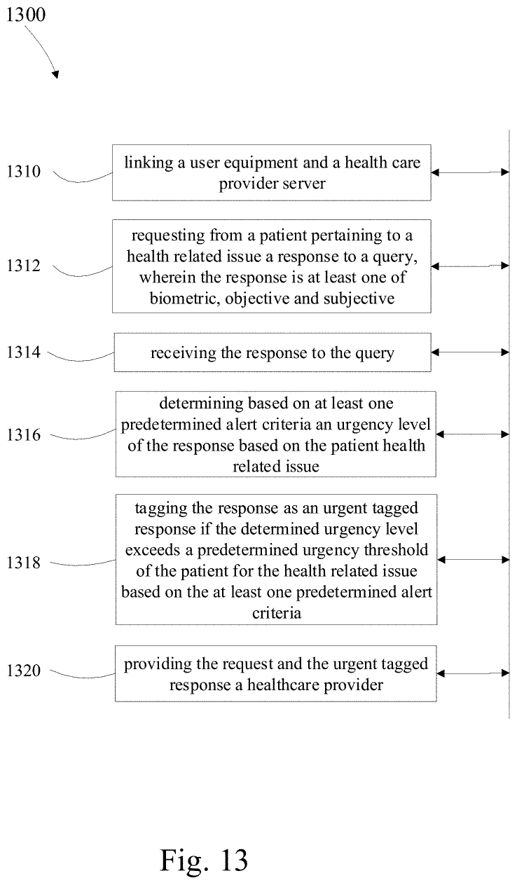

FIG. 14 illustrates an example onboarding session with a patient, according to example embodiments of the present application.

FIG. 15 illustrates example questions asked of a patient, according to example embodiments of the present application.

FIG. 16 illustrates an example of the layout of the questions asked by the system, according to example embodiments of the present application.

FIG. 17 illustrates an example of the files used to program the system are placed, according to example embodiments of the present application.

FIG. 18 illustrates an example Excel file with some information for the hypertension and weekly blood pressure journey, according to example embodiments of the present application.

FIG. 19 illustrates another example Excel file with some information for the hypertension and weekly blood pressure journey, according to example embodiments of the present application.

FIG. 20 illustrates an example of message texts associated with the hypertension and weekly blood pressure journey, according to example embodiments of the present application.

FIG. 21 illustrates a portion of the Excel form and indicates answers to questions, according to example embodiments of the present application.

FIG. 22 illustrates an example of guardrail values and their possible resetting by medical personnel, according to example embodiments of the present application.

FIG. 23 illustrates a review chart having the most critical patients sorted by criticality, according to example embodiments of the present application.

FIG. 24 illustrates a review of the topmost critical patient, according to example embodiments of the present application.

FIG. 25 illustrates environmental condition questions that may have an impact on health issues, according to example embodiments of the present application.

FIG. 26 illustrates example questions related to spreading illnesses such as COVID-19, according to example embodiments of the present application.

FIG. 27 illustrates example questions related to spreading illnesses such as COVID-19, according to example embodiments of the present application.

FIG. 28 illustrates an example of a hypertension and weekly blood pressure journey message flow for different blood pressures, according to example embodiments of the present application.

### DETAILED DESCRIPTION

It will be readily understood that the components of the present application, as generally described and illustrated in the figures herein, may be arranged and designed in a wide variety of different configurations. Thus, the following detailed description of the embodiments of a method, apparatus, and system, as represented in the attached figures, is not intended to limit the scope of the application as claimed, but is merely representative of selected embodiments of the application.

The features, structures, or characteristics of the application described throughout this specification may be combined in any suitable manner in one or more embodiments. For example, the usage of the phrases “example embodiments”, “some embodiments”, or other similar language, throughout this specification refers to the fact that a particular feature, structure, or characteristic described in connection with the embodiment may be included in at least one embodiment of the present application. Thus, appearances of the phrases “example embodiments”, “in some embodiments”, “in other embodiments”, or other similar language, throughout this specification do not necessarily all refer to the same group of embodiments, and the described features, structures, or characteristics may be combined in any suitable manner in one or more embodiments.

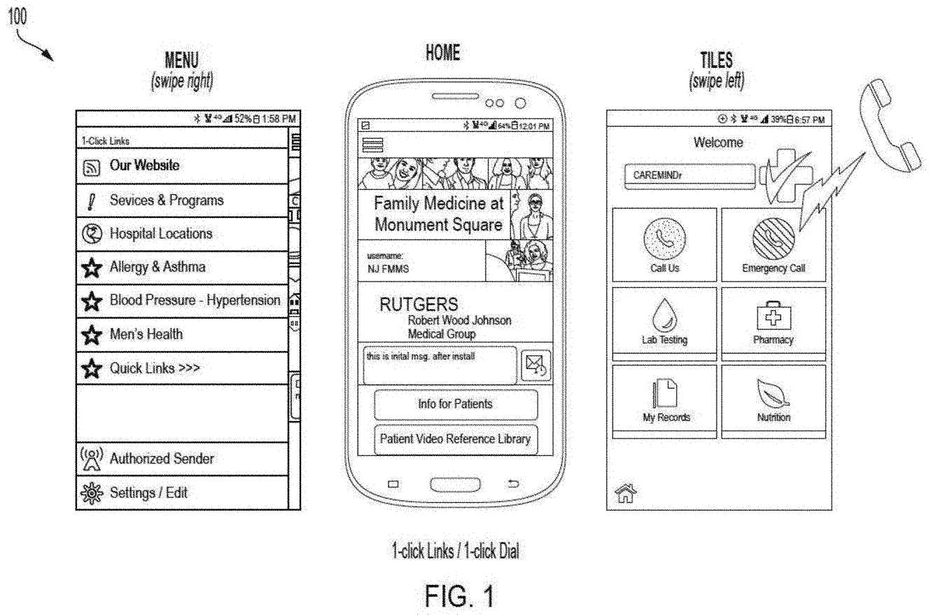

FIG. 1 illustrates an example of the integrated application platform according to example embodiments. Referring to FIG. 1, the configuration 100 includes a menu user interface, a home user interface and a set of option tiles for accessing third party resources, such as test results, emergency concerns, pharmacy information, etc.

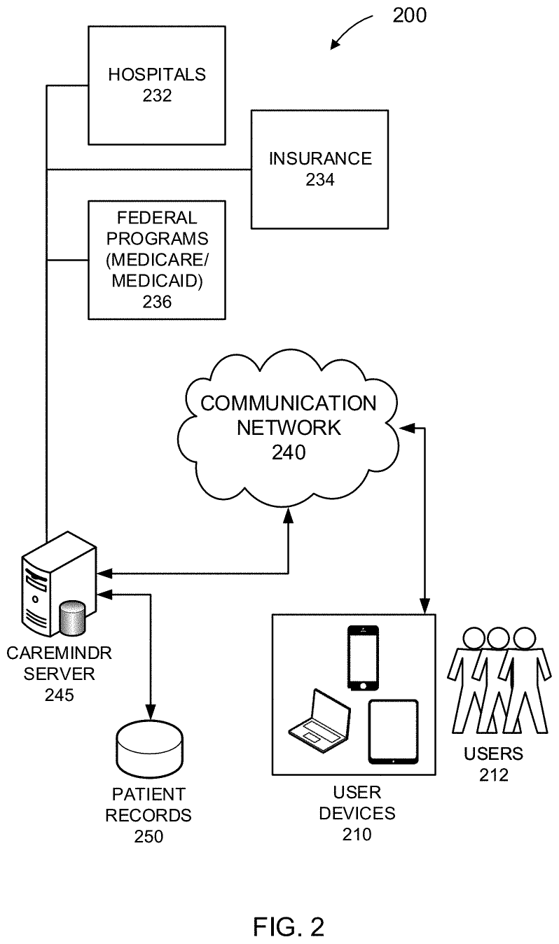

FIG. 2 illustrates a network configuration of the third party participants of the integrated application according to example embodiments. Referring to FIG. 2, the network includes a central server 245 with patient records 250. The information needed to provide treatment plans and other integrated services may require access to hospital and other provider services 232, insurance company information 234, drug providers, federal program administrators 236, etc. The information may be incorporated into any treatment plan or other integrated service model accessed by a user device 210 operated by a user 212. The servers and third party modules may operate on-site or in a cloud network managed by the providers.

Examples of treatment plans and other objectives may include a care management service for assessment of patient medical needs. The system and application may ensure timely receipt of all recommended treatment actions, drugs, third party services and over a designated period. Also, referrals to other providers and additional services may provide emergency visits, discharge instructions, nursing facility operations, and home health care functions. In operation, the procedure may begin with the medical treatment provider creating a treatment plan or ‘journey’ for each patient. Each journey is generally for a single chronic condition or objective. One patient may have multiple journeys integrated into a single application. Also, the journeys may originate from various providers and service entities. The journey will provide the healthcare provider with biometric, objective and subjective data to enable evidence-based medical decisions. As an example, the biometric data may be glucometer data collected from a blue tooth enabled device and made available to the physician, objective data such as whether the patient visited an emergency room or hospital and subjective data such as how the patient is feeling.

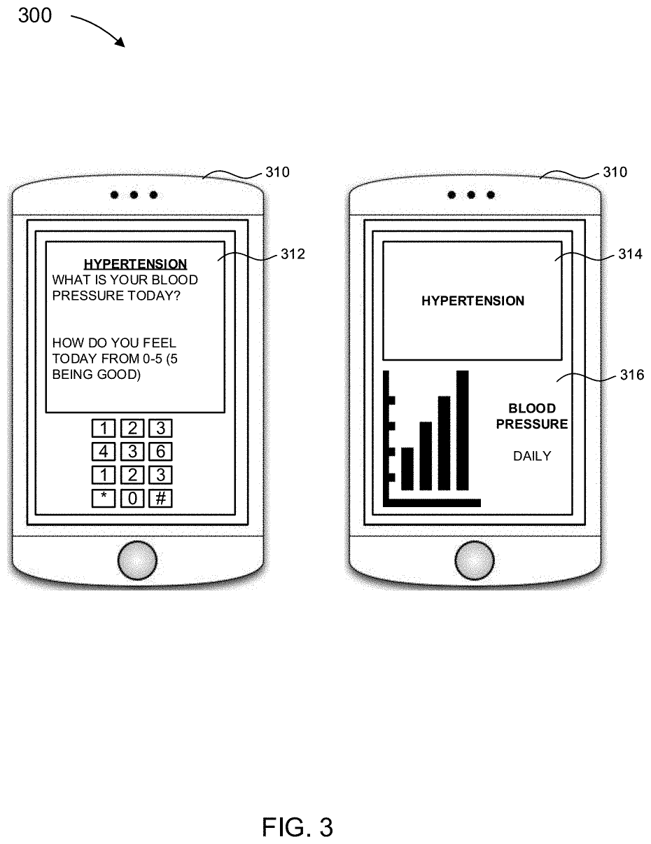

FIG. 3 illustrates a user smartphone interface of an example treatment plan according to example embodiments. Referring to FIG. 3, the journey for “hypertension” may have been created or modified by a patient doctor and may include an interface 300 with a smartphone device 310 and a screen option configuration providing questions 312, information about the treatment, reminders and other functions. The example in FIG. 3 provides for a set of questions 312 and a journey topic 314 along with a graph of blood pressure records 316 as measured over time from various interactions.

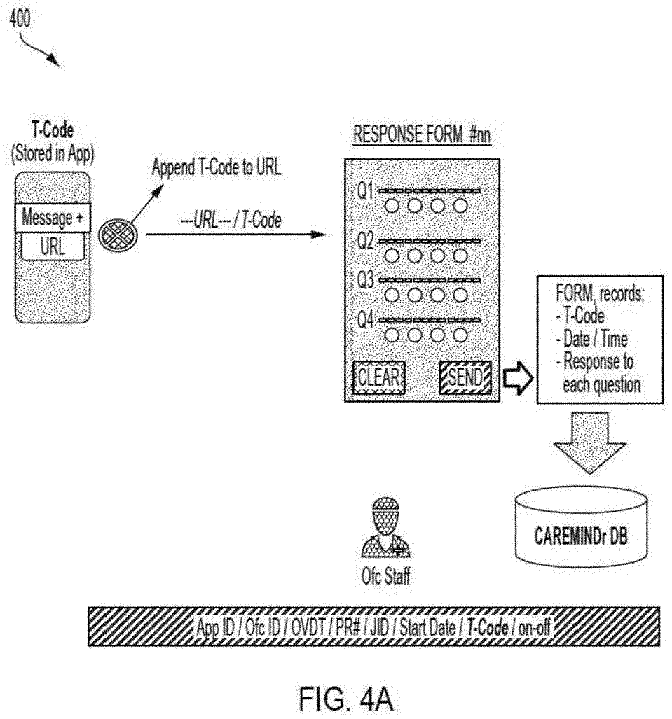

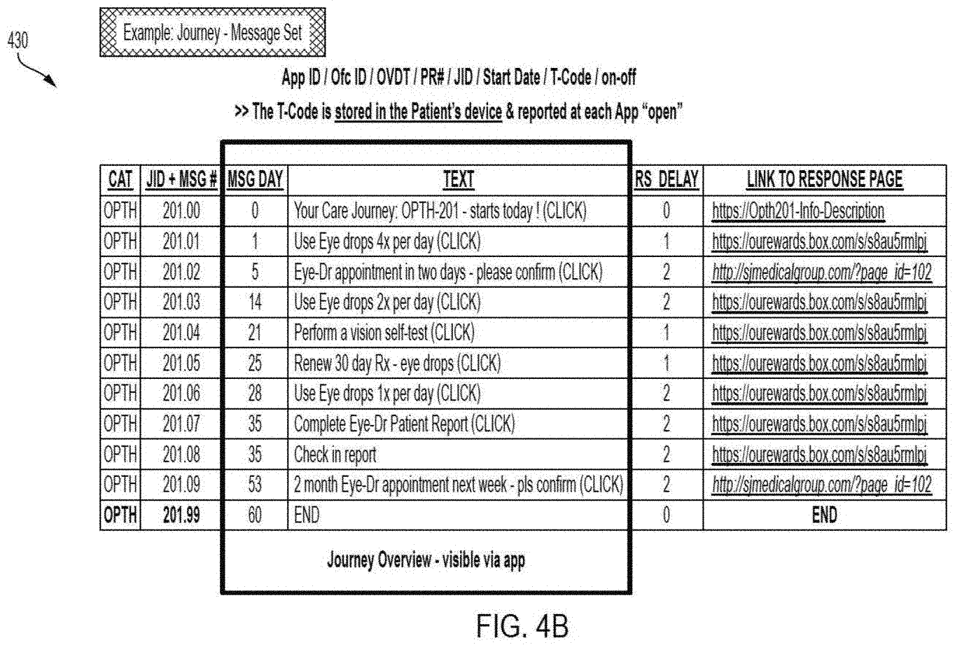

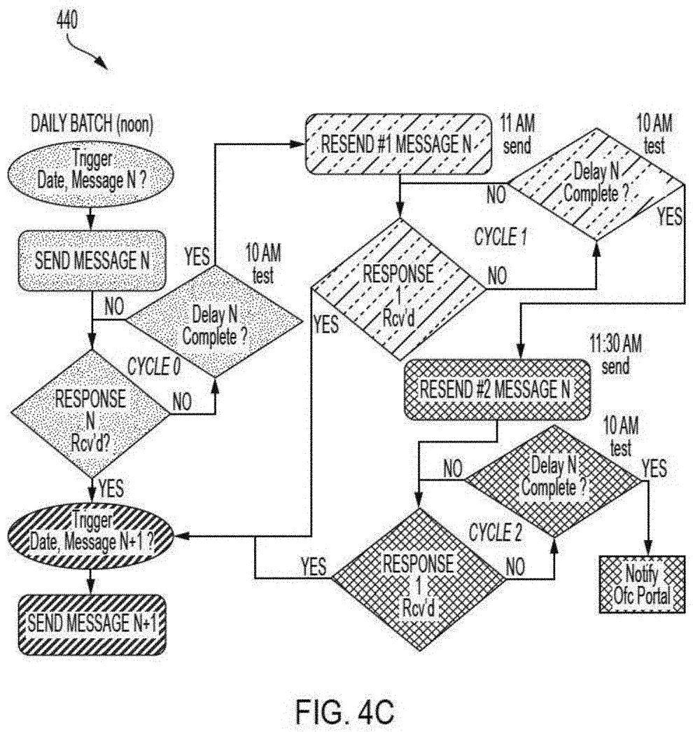

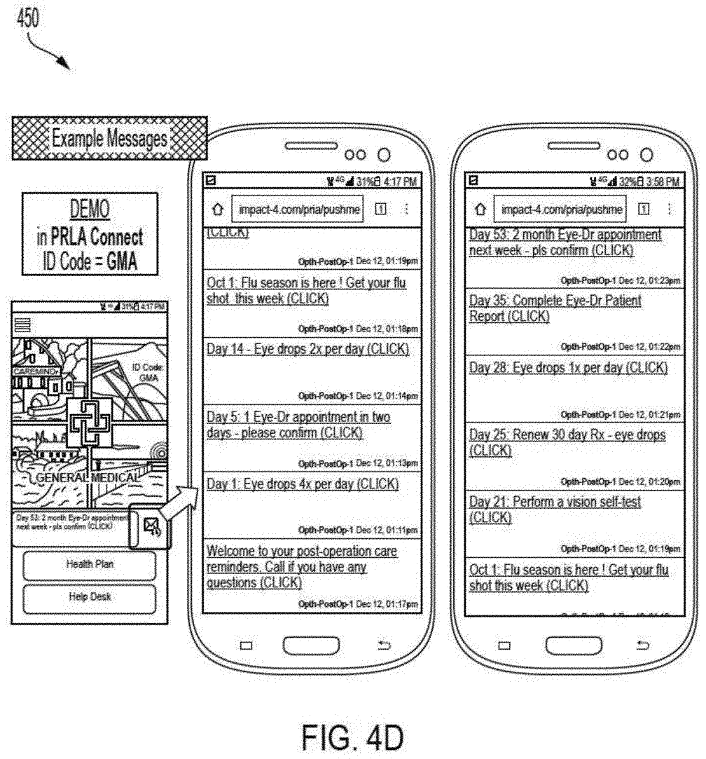

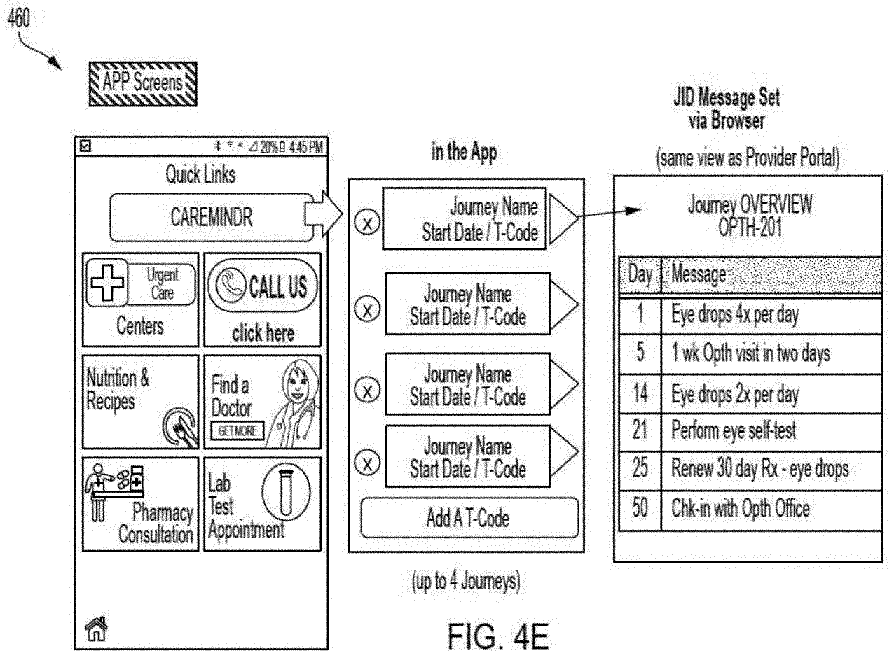

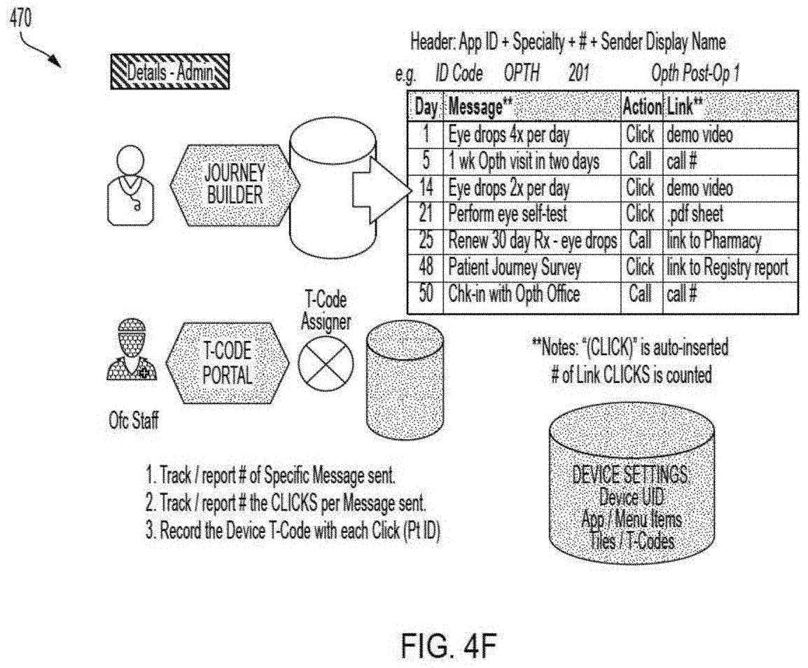

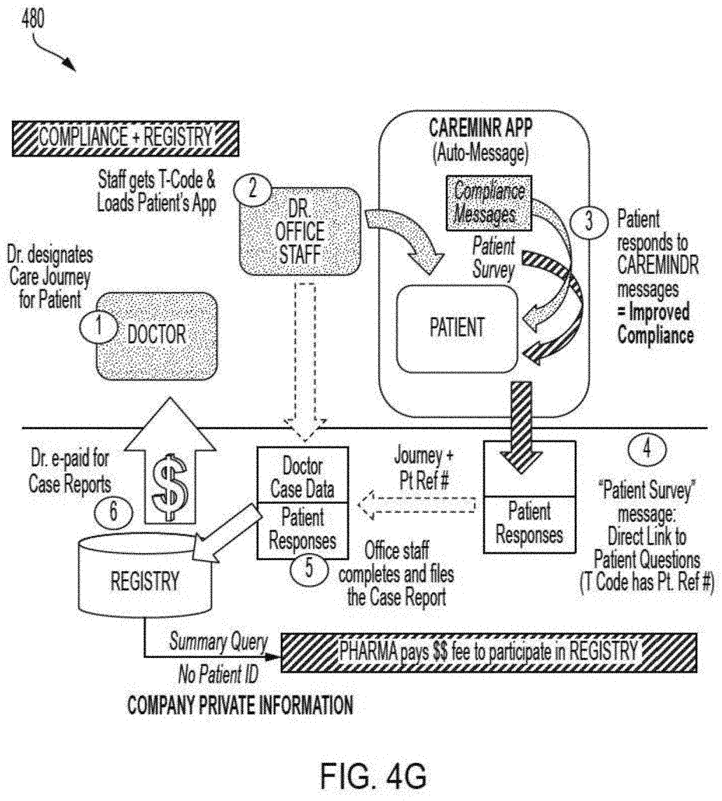

FIG. 4A illustrates an example setup configuration for a new course of treatment according to example embodiments. Referring to FIG. 4A, the illustration 400 includes the basic setup functions of linking a particular journey T-code (unique code) to the message and/or universal resource locator (URL) to link the application of the user to a customized template, such as a response form, questionnaire, etc. The unique T-code, date, time, response, and other records for each instance may be stored in a patient record managed by the application system database.

FIG. 4B illustrates an example database entry for the new course of treatment according to example embodiments. Referring to FIG. 4B, the example configuration 430 includes a database entry of messages which are organized by a category, in this case ophthalmology, and with a message content, including a link to a response page. The context and add-ons of a particular message may be customized based on a preferred layout or a default layout.

FIG. 4C illustrates a flow diagram 440 configuration for the new course of treatment according to example embodiments. Referring to FIG. 4C, the flow diagram includes a daily batch of messages which are setup to be delivered to one or more assigned patients. The process begins with a trigger to send a message, such as a matured date or time. The process then continues to deliver additional messages once confirmation of delivery is made. If the message is delayed or the response required is not received, the message may be resent as a late message requiring immediate attention. The process may continue to cycle to identify whether any messages are outstanding or have not been confirmed.

FIG. 4D illustrates an example list of messages for the ongoing course of treatment according to example embodiments. Referring to FIG. 4D, in this illustration 450, the various messages intended for a particular patient are illustrated by date. FIG. 4E illustrates an example setup configuration for various courses of treatment according to example embodiments. Referring to FIG. 4E, the configuration 460 includes a menu of options along with a set of potential journeys the user may be assigned to manage the ongoing health care treatment plans for that user. The overview of treatment options and dates are included for reference purposes.

FIG. 4F illustrates an example set of details of an ongoing course of treatment according to example embodiments. Referring to FIG. 4F, the details of the administrator are shown to include a journey builder function based on certain parameters, such as an identification code, specialty, a number and a sender name. The number of messages, responses and actions are recorded to demonstrate the user's interaction with the application and the specific treatment plan(s).

FIG. 4G illustrates an example network configuration of the various third parties involved in the application operation and compliance according to example embodiments. Referring to FIG. 4G, the large-scale network of communications among the integrated platform 480 demonstrates the process initiating with the doctor's office establishing a journey for the patient and assigning a T-code (unique code). The patient's responses are identified along with links and references to third party message links and other information sources.

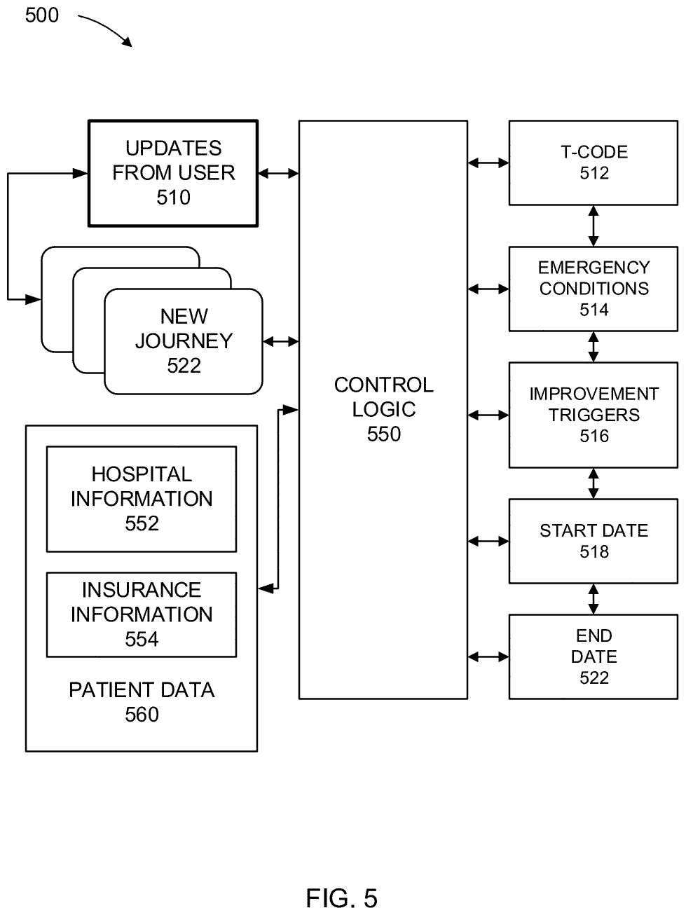

FIG. 5 illustrates a logic module configured to process the input and output parameters of the application according to example embodiments. Referring to FIG. 5, the control logic platform 500 includes a control logic unit 555, such as a processor or other processing entity that may receive updates from a user 510, new journey information 522 and/or patient data 560 including hospital 552, insurance 554, and other information. The logic may be configured to identify and link the unique T-code 512, emergency conditions 514, improvement triggers 516 for optimal changes to the treatment plan, along with dates 518 and new journey information 522 to perform the treatment plan.

In addition, while the term “message” has been used in the description of embodiments of the present application, the application may be applied to many types of network data, such as, packet, frame, datagram, etc. For purposes of this application, the term “message” also includes packet, frame, datagram, and any equivalents thereof. Furthermore, while certain types of messages and signaling are depicted in exemplary embodiments of the application, the application is not limited to a certain type of message, and the application is not limited to a certain type of signaling.

According to example embodiments, a user device, such as a smartphone, cellular phone, tablet device, laptop or other computing device with a memory and processor, may communicate with another computing device and/or a server to provide an integrated communication platform.

Example embodiments provide a computer system programmed to use automated messaging from medical offices to specific patients. The application is not limited to medical procedures and functions and may be used with other configurations for various purposes and services benefitting the end user. Example embodiments include three main computer systems, which work together in an integrated manner including a management platform that controls set-up, functionality, activity reporting, and messaging credentials for the users. An administrative platform which the doctor and doctor's office can access via the internet, and a mobile application that a patient can download into a mobile computer device such as a smartphone or tablet. The management platform acts as the nexus of the system sending outgoing messages on behalf of the healthcare provider and forwarding patient responses to the healthcare provider's administrative platform. The medical office may have a specific identification that is stored within the management platform.

The integrated platform provides a way of checking-in with a patient at prescribed intervals during times between office visits and when undergoing certain treatment that the doctor is providing or overseeing for the patient. The patient dialog may gather relevant information about the status of the patient's conditions or recovery and can be modified or tailored to specifically meet the dialog requirements of the treating physician. Once initiated by the doctor's office, the application operates in an autonomous manner by delivering messages to the patient to prompt responses if needed. The application functions are monitored to assure that the patient replies to the information requests from the doctor, otherwise a no-response alert is sent to the doctor's office. The interactions are recorded and time-stamped, providing an auditable record of the dialog, suitable for insurance billing purposes. The application can also support biometric information from devices that measure certain body functions, such as diabetes glucometers, or blood pressure cuffs, or any sensory readable health care metric. The application may also create a longitudinal record of information for the patient to illustrate week-to-week trends.

Response Alert Tags

As has been stated earlier, this method and system is utilized when a patient visits a healthcare provider for an illness/condition which is diagnosed and treated. The treatment occurs over a period and is referred to as a journey. The system tracks a patient's progress along the journey for that illness or condition and solicits health information from the patient at clinically relevant intervals, across an extended time period to enable evidence based medicine. The specific information sought, the intervals, and the time period duration apply to specific conditions or illnesses for which a specific patient is being treated.

This solicitation for patient information may take the form of queries sent to the patient for information when the responses to those queries are delivered to the patient's healthcare provider (e.g. physician). The patient's journey may have several waypoints occurring at the clinically relevant intervals. The responses to the queries at these waypoints are meant to determine a patient's progress and status and to present to the healthcare provider evidence upon which to conduct evidence-based medicine. The responses are collected by the system and measured against historical norms for the patient and/or expected answers for similar patients on similar journeys.

In the event of an unexpected response to a query at that waypoint, the response is treated as notable. Notable events may be considered non-urgent or may be considered urgent or emergent. This divergence from the expected response outcome is graded for severity or urgency. If the severity or urgency of the response exceeds a predetermined threshold for that patient for that journey for that illness or condition at that waypoint, an urgent tag is created and sent to the healthcare provider. The grading may be one of an immediate medical action advisory, a follow-up advisory and a medical history review advisory

The information requested in the query is sent in a structured format to allow ease in answering and the response data is delivered to the healthcare provider in a structured data format to facilitate ease in analysis and trend detection.

The response alert tag is a feature that “tags” certain responses provided by the patient as information that requires follow-up or special notice by the patient's healthcare provider. The tag may indicate a level of severity or urgency, thus alerting the provider to information that may need immediate medical action, additional follow-up with the patient or a specific review of the patient's medical history.

The tag may be communicated to the provider through multiple channels depending upon circumstance and urgency and in an immediate manner or in a weekly aggregated format depending in part upon urgency.

Workflow instructions may be electronically linked to a tag, so that the specific healthcare provider that reviews the data will have guidance about the actions to be taken when a tag appears and any escalation of clinical review that might be appropriate.

Each patient for each illness or condition is interacted with by the system at intervals which are relevant to that illness or condition and the queries are sent to determine the patient's progress or status. The received response to the query is measured against an expected response, and anomalies or offsets are noted. If these response anomalies or offsets are larger than a predetermined amount, an urgent or severe issue may need to be addressed. Thus, the response is tagged as an urgent tagged response and may be sent utilizing a priority delivery schedule, a priority delivery indicia on the response and may be made to a priority delivery list determined by the healthcare provider. The response may be tagged as non-urgent if the determined urgency level does not meet the predetermined urgency threshold of the patient for the health-related issue.

The structured format allows an overlap of queries so that the patient is not answering multiple identical queries at any one point in time. Additionally, the structured format allows the data to be collected and logged in a structured format and assembled for future review both by the practitioner and the patient to determine trends.

In one example a method, includes requesting via a cloud-based system from a patient response to a query and receiving the response to the health-related query, determining an urgency level of the response based on the patient health-related issue and tagging the response as an urgent tagged response if the determined urgency level exceeds a predetermined urgency threshold of the patient for the health-related issue.

The method also includes providing the urgent tagged response to the health provider, where the urgent tagged response may be sent utilizing a priority delivery schedule, a priority delivery indicia for the response and may be made to a priority delivery list.

The method may also include tagging the response as non-urgent if the determined urgency level does not meet the predetermined urgency threshold of the patient for the health-related issue.

If the determined urgency level of the response is such that it rises to the level of a medical emergency, then the primary care physician may be immediately notified as well as emergency services such as 911 and if deemed appropriate, dispatched to the location identified either by the patient or gathered from a location indicator in his mobile device. If the response is deemed critical, in situations where the primary physician is not immediately available, an emergency medical specialist may be placed in active direct communication with the patient. The system would make available to the first responder the query and response to provide context for the escalation.

The response may be graded as to the tagged urgency level of the response, where the grading is at least one of an immediate medical action advisory, a follow-up advisory and a medical history review advisory. A follow-on query may be sent based on the urgent tagged response to give the provider context to the urgent tagged response. As an example, if the patient responds that they have been to the emergency room (ER) that may trigger another set of queries about the ER visit to add context to the response. This second set of queries may determine whether the ER visit was related to conditions or illnesses related to the journey, or whether visit was for a condition unrelated to the journey, but still of interest to the healthcare provider.

In another example a cloud-based system links a mobile device and a healthcare provider server. The cloud-based system requests a response to a query from a patient pertaining to a health-related issue, receives the response to the query and determines an urgency level of the response based on the patient health-related issue. The system also tags the response as an urgent tagged response if the determined urgency level exceeds a predetermined urgency threshold of the patient for the health-related issue and provides the urgent tagged response to health provider.

The cloud-based system may receive via the mobile device a sensor signal provided by a medical device in response to the query. The medical device may be a blood pressure monitor, a glucometer, a pulse meter, a continuous positive airway pressure device, a heart monitor, an implanted medical device and the like.

The cloud-based system may receive via the mobile device an audio or text message indicating a medical distress condition in response to the query or may overhear the patient indicating a medical distress condition in conversations or texts in an unsolicited message.

The system may also interpret patient actions in regards to patient historical norms, such as, if the patient is overheard slurring his speech, he may be having a stroke, or if he is discussing that he has pressure in his chest or his left arm is numb, he may be having a heart attack. At this point the system may connect him directly to a medical specialist and take other appropriate action, such as determining his location and dispatching emergency services.

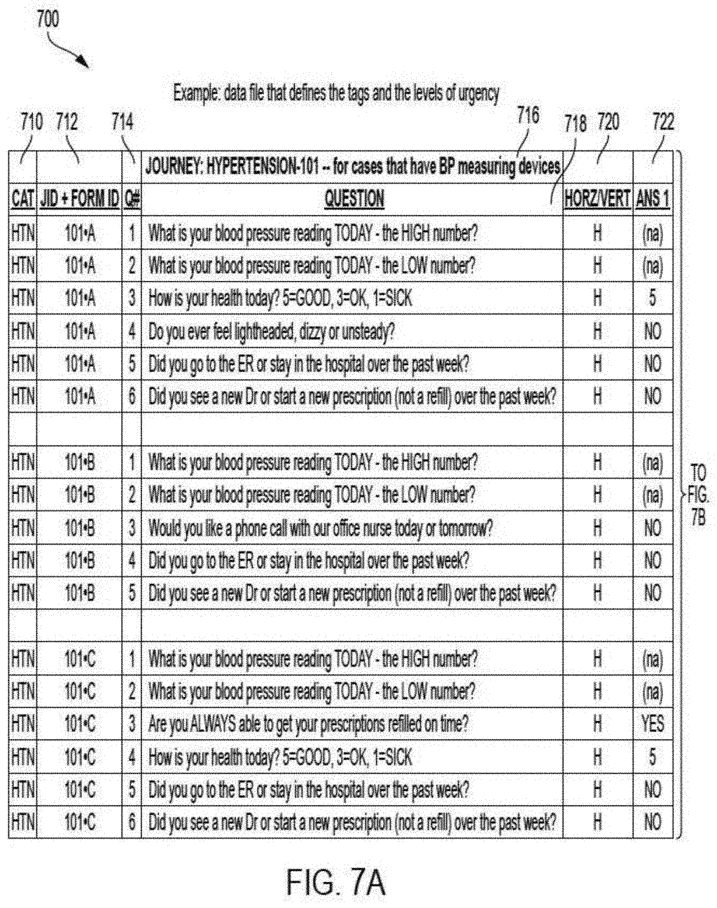

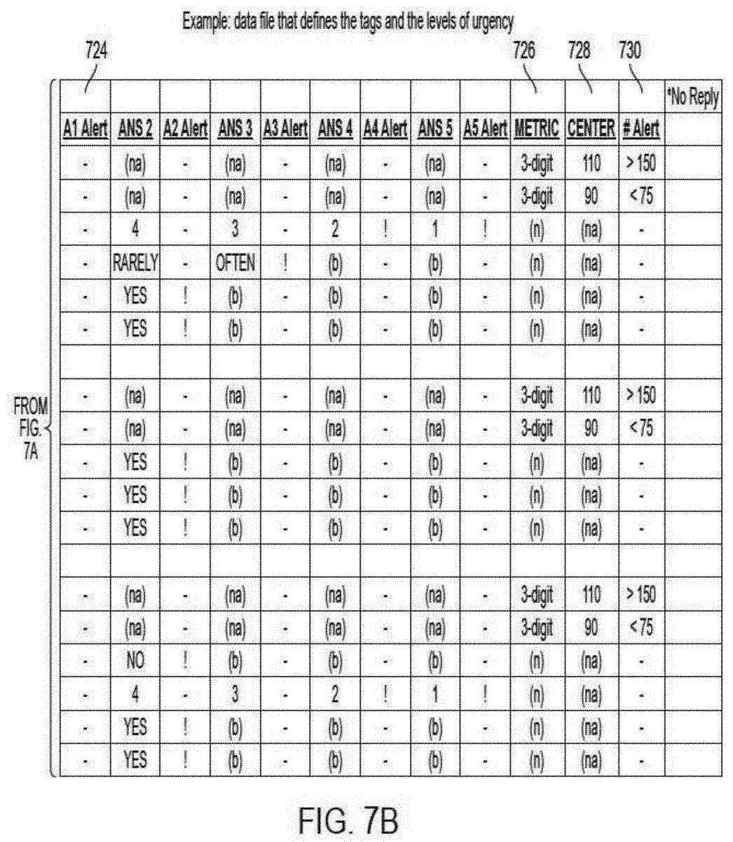

FIGS. 7A and 7B depicts an example data file 700 that defines the tags and levels of urgency. The parts of the data file include a category of question 710, the journey ID and form ID 712, a question number 714, a patient journey for that specific illness or condition 716 and questions associated with that journey 718. The data layouts may be shown horizontally or vertically 720 which allows viewing of individual answers to queries. The historical and current query response 722 and the determined alert 724 are shown, which in a historical context allows detection of trends. The data output of the metric 726 may be numerical, yes or no, and the like, a median query answer 728 for that patient or that condition and an alert threshold 730 which may be modified by the healthcare profession are shown.

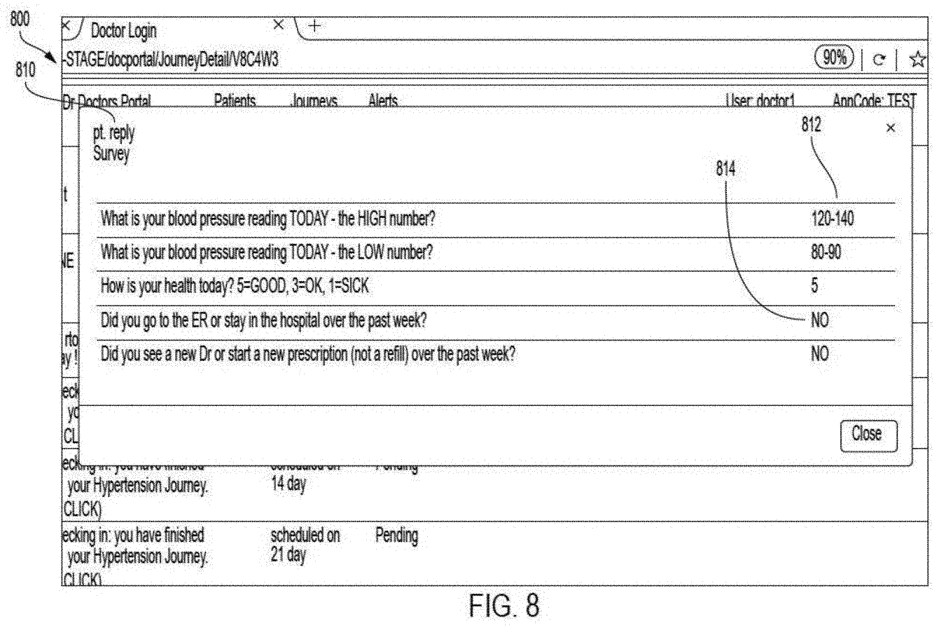

FIG. 8 depicts a patient survey reply 810 having a band of expected replies for blood pressure 812, how the patient feels and whether he went to the emergency room, hospital 814 or has started a new prescription.

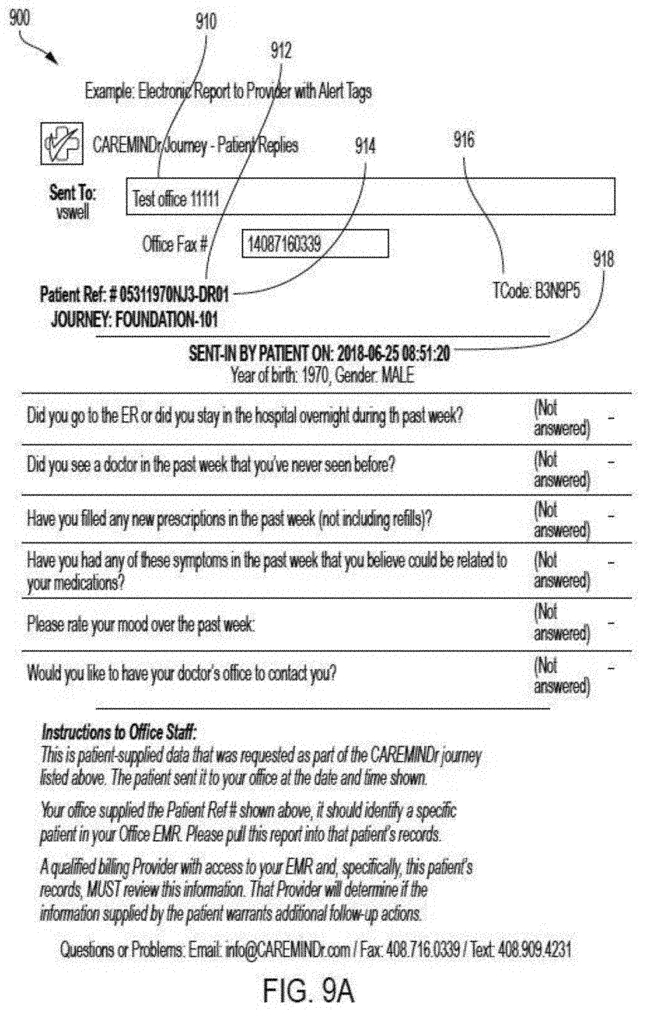

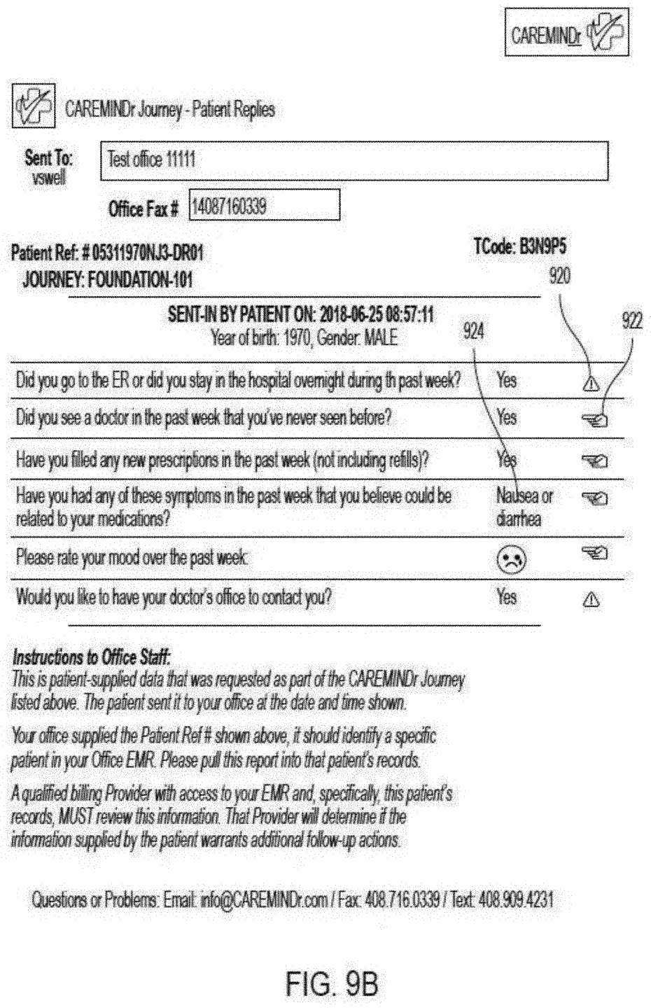

FIGS. 9A and 9B depicts both a non-responsive and responsive reply set 900. The recipient 910, patient reference number 912, journey ID 914, unique T-Code 916 and timestamp 918 are depicted in the reply. A responsive report having alerts indicates an urgent issue 920, a follow up item 922 and an emoticon 924 indicating a patient feeling is shown.

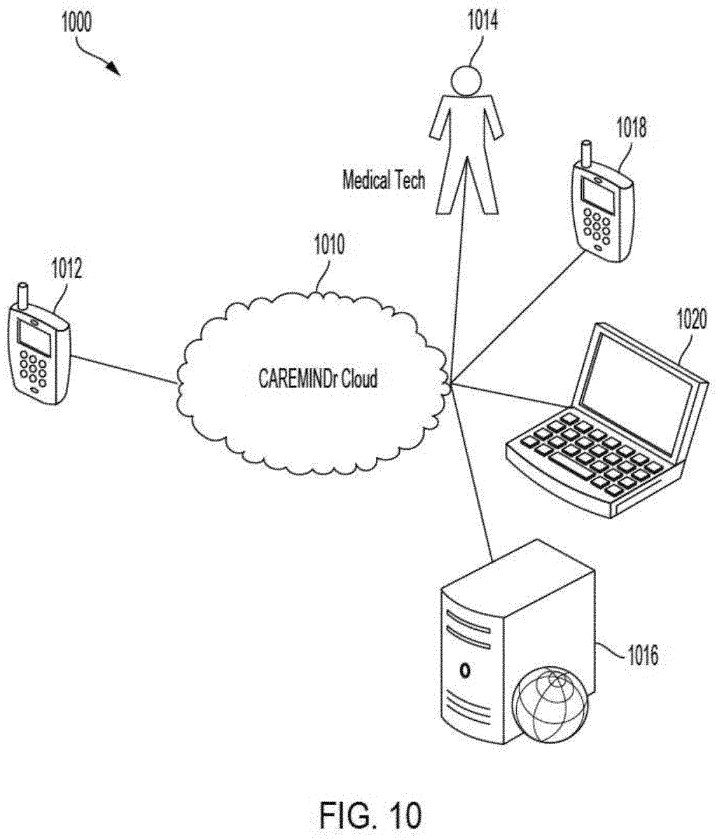

FIG. 10 depicts an example 1000 of communication associated with the alert tag. The cloud-based system 1010 sends a request with queries to the patient's communication device 1012 which the patient fills out and returns. The cloud-based system 1010 reviews the response and determines whether there are urgent or emergency issues and sends an urgent tagged response to the healthcare provider.

If there is an emergency issue the cloud-based system may contact or place the patient in contact with a medical technician 1014 in addition to notifying the healthcare provider by means of the healthcare provider's server 1016, the cloud-based system may issue a text or message to the healthcare provider. The communication route from the healthcare provider may be by means of mobile device 1018, computer 1020 or the like. The cloud-based system may directly connect the patient via to the patient's communication device 1012 to the healthcare provider under appropriate circumstances. Non-urgent issues are sent to the healthcare provider for later review.

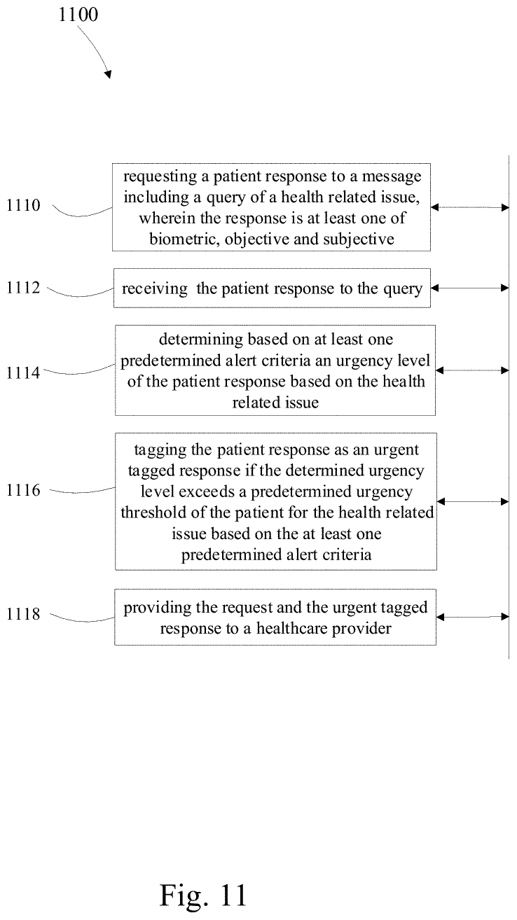

A second example method is shown in FIG. 11 related to response tagging, may include requesting 1110 a patient response to a message including a query of a health-related issue, wherein the response is at least one of biometric, objective and subjective and receiving 1112 the patient response to the query. The method then provides determining 1114 based on at least one predetermined alert criteria an urgency level of the patient response based on the health-related issue, tagging 1116 the patient response as an urgent tagged response if the determined urgency level exceeds a predetermined urgency threshold of the patient for the health-related issue based on the at least one predetermined alert criteria and providing 1118 the request and the urgent tagged response to a healthcare provider. The method may also include proposing a set of workflow instructions linked to an urgent or non-urgent tagged response and presenting a clinical escalation review advisory if the response is tagged as an urgent tagged response.

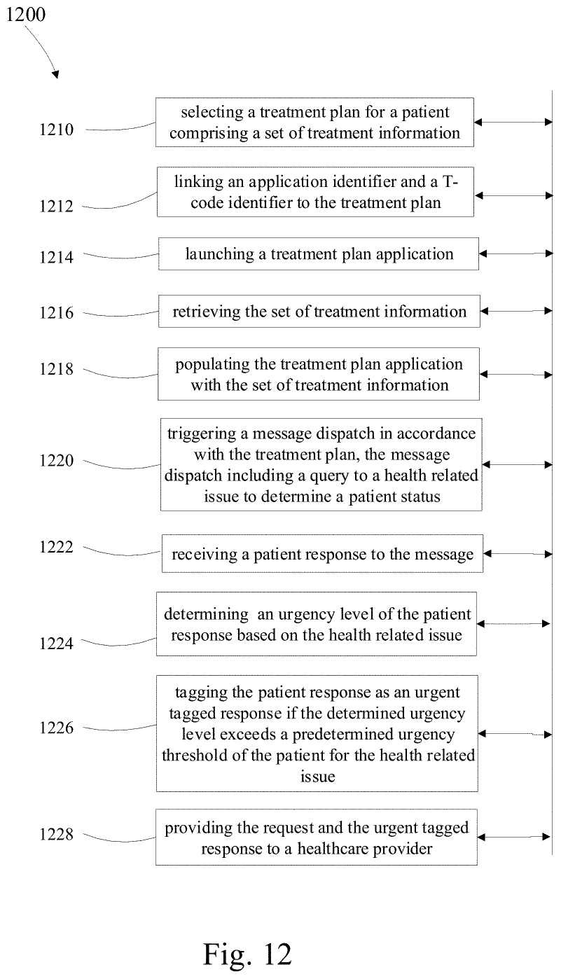

A first example method shown in FIG. 12, 1200, may include, selecting 1210 a treatment plan for a patient comprising a set of treatment information, linking 1212 an application identifier and a T-code identifier to the treatment plan and launching 1214 a treatment plan application. The method further includes retrieving 1216 the set of treatment information, populating 1218 the treatment plan application with the set of treatment information and triggering 1220 a message dispatch in accordance with the treatment plan. The message dispatch includes a query to a health-related issue to determine a patient status and the system receives 1222 a patient response to the message. The method then provides determining 1224 an urgency level of the patient response based on the health-related issue, tagging 1226 the patient response as an urgent tagged response if the determined urgency level exceeds a predetermined urgency threshold of the patient for the health-related issue and providing 1228 the request and the urgent tagged response to a healthcare provider. The method may also include proposing a set of workflow instructions linked to an urgent or non-urgent tagged response and presenting a clinical escalation review advisory if the response is tagged as an urgent tagged response.

With respect to the timing of patient responses, the first example method may also include, awaiting the patient response to the message for a late response duration and categorizing the patient response if the patient response is received within the late response duration. If the patient response is not received within the late response duration the method further comprises sending a duplicate message and flagging the patient response as non-responsive if the patient response to the duplicate message is not received within a second late response duration.

The timing of the message dispatches associated with the treatment plan is partly governed by a trigger table. The method may include loading the trigger table having a set of trigger dates based on the treatment plan where the message dispatch is sent according to the set of trigger dates. The method may further include receiving a message start date and receiving an initialization message from a patient mobile device to initiate the treatment plan and to initialize the set of trigger dates in the trigger table.

A first example non-transitory computer readable medium 1300 comprising instructions associated with the tagging of responses that, when read by a processor, cause the processor to perform; linking 1310 a mobile device and a health care provider server, requesting 1312 from a patient pertaining to a health-related issue a response to a query, wherein the response is at least one of biometric, objective and subjective and receiving 1314 the response to the query. The processor then determines 1316 based on at least one predetermined alert criteria an urgency level of the response based on the patient health-related issue, tags 1318 the response as an urgent tagged response if the determined urgency level exceeds a predetermined urgency threshold of the patient for the health-related issue based on the at least one predetermined alert criteria and provides 1320 the request and the urgent tagged response a healthcare provider.

Most medical oversight is provided post-event in which a patient has been identified and is either currently in the throes of a medical condition or is recovering from a medical condition. In this example, a single patient is looked after by a group of people, this is a resource rich environment in which one patient has the attention of the medical community.

The issue that this application may provide a solution to is one that provides medical oversight to a large community, in essence to act as a fire lookout tower in a forest. In the case of a fire lookout tower, a small number of people are looking after a vast number of trees. When a problem is detected, the fire fighters are sent to that part of the forest to prevent a larger breakout. This solution is the medical analog to that illustration. In the medical case, we have a small number of highly trained personnel looking after an entire community. On a per person basis, this is a very medical resource restricted environment.

The two different environments have similar but different focuses. In a resource rich environment, the viewing equipment used is the equivalent of a microscope, whereas, in a resource restricted environment the viewing equipment utilized are binoculars. Although the medical questions asked may share a foundational basis, how those questions are asked and how the responses are analyzed fall along completely different tracks.

The COVID-19 pandemic has brought to the surface medical attention misallocation issues present in the current state of medical care. One issue that has been highlighted is overtreatment and unnecessary treatment of patients, including office visits based on the calendar rather than current health needs, which is also described as low value care. Additionally, the pandemic has focused attention on elective procedures, which has subsequently shown that the performance of the elective procedure has had no demonstrable beneficial effect or reduced the recurrence of subsequent doctor visits. This low value care may be defined as medical actions in which the potential for harm far outweighs possible beneficial effects. Low value care also uses up doctor time and appointments for patients that do not require immediate attention, while not scheduling patients with immediate needs, mostly because of a lack of awareness.

Another highlighted misallocation of medical attention has been detected in the current fee for service (FFS) model of medical payment—doctors are paid for specific work they do, unrelated to whether it is appropriate to a current patient need. While this FFS model, which requires doctors to perform certain tasks in certain very specific ways, may function in an age of a medically rich resource environment, it does not function well at all when providing community health services for many patients.

The present potential solution goes to the heart of the current medical misallocation issue, by using an automated approach for periodically checking on and assessing all patients with a specific condition. This process will find that the majority of patients are fine and require no attention at this time, and it focuses medical attention on those patients that require it. The potential solution allows a resource sparse medical team to leverage the patient's inputs in conjunction with medically programmed and adjusted logic to direct focus on those patients needing immediate care, while continually keeping an eye on the remainder of the patient pool.

The questions in this possible solution are specific to the patient's condition, the responses may be numeric or multiple choice and may be biometric, objective, subjective and requests. A biometric question would be one in which a number such as diastolic and systolic blood pressure are input, or a blood glucose level is input. An objective question pertains to facts such as “have you filled any new prescriptions in the past week?”, the answer of which is fact based and does not require patient interpretation or analysis. A subjective question would be one such as “please rate your mood over the last week” or “have you lost the sense of taste or smell over the last week?”. A request would be worded such as “would you like your doctor to contact you?”

The interpretation of the results is assembled in an array format that may be adjusted by the patient's doctor. The medical personnel in charge of the patient has the final say in how a response is interpreted and the medical weight given to the response. The triage is very simple, the highest alert level is the weight of the response, based on the input table any number or physician assigned emergency, critical, warning or nominal and tagged as such.

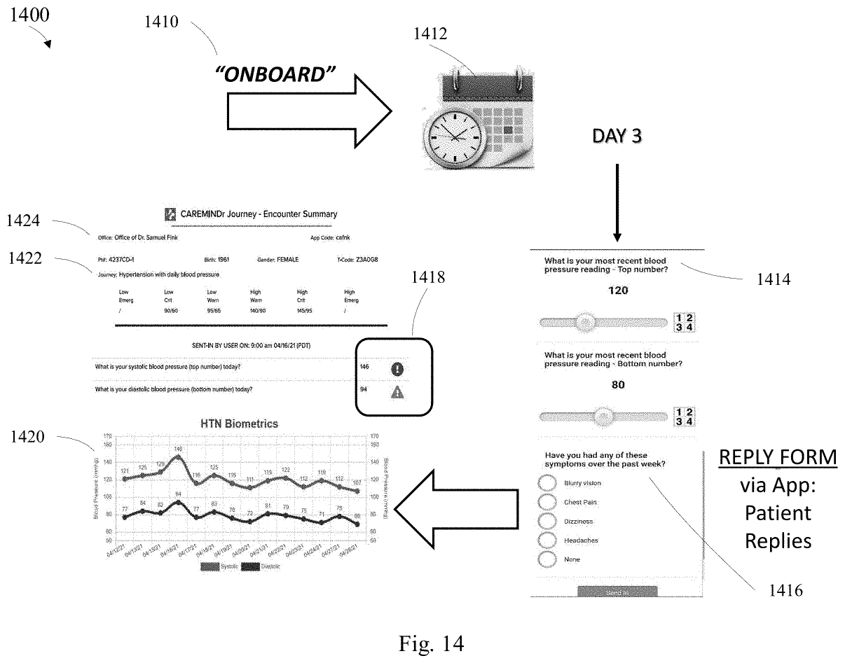

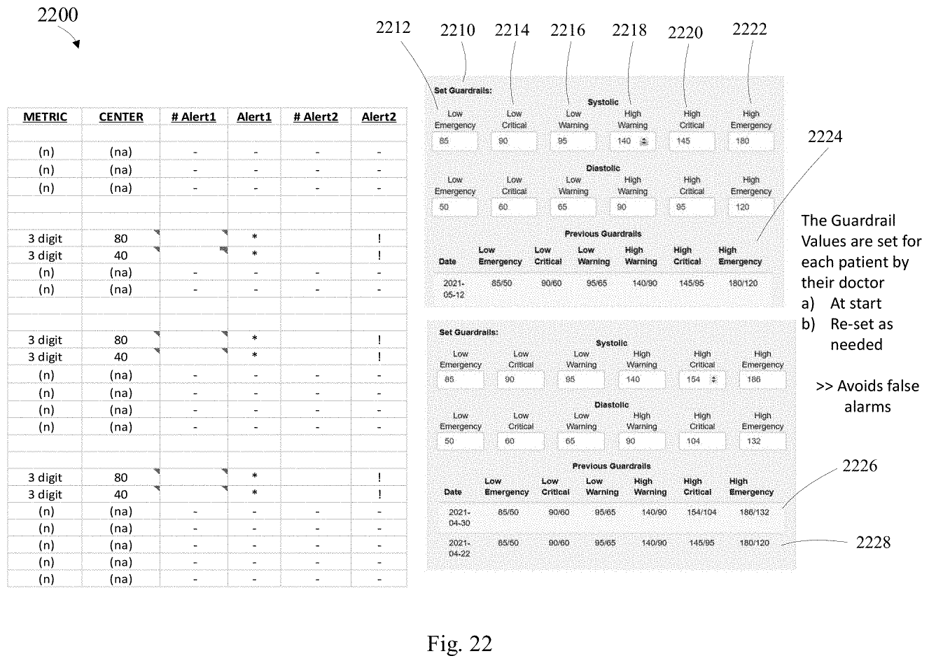

FIG. 14 depicts an example onboarding session with a patient. The patient data is loaded to the system 1410 and a calendar is set up 1412 in this case for a journey for hypertension with daily blood pressure checks 1422 for a patient of Dr. Samuel Fink 1424. There are a series of questions on the journey and on day 3 the patient is asked what is their most recent blood pressure top number, systolic, and bottom number, diastolic, 1414, this would be described as biometric data as it is acquired from a device of some type. The patient is also asked if they have had any symptoms over the last week 1416, this would be considered an objective question. The patient's blood pressure readings are tallied and if the systolic or diastolic blood pressures is concerning, then they are flagged according to their seriousness 1418 and a biometric chart is provided for the healthcare provider 1420. The exact criteria for an emergency, critical or warning for both systolic and diastolic blood pressure is shown in FIG. 22. These numbers are referred to as guardrails may be reset by the medical staff to avoid false alarms.

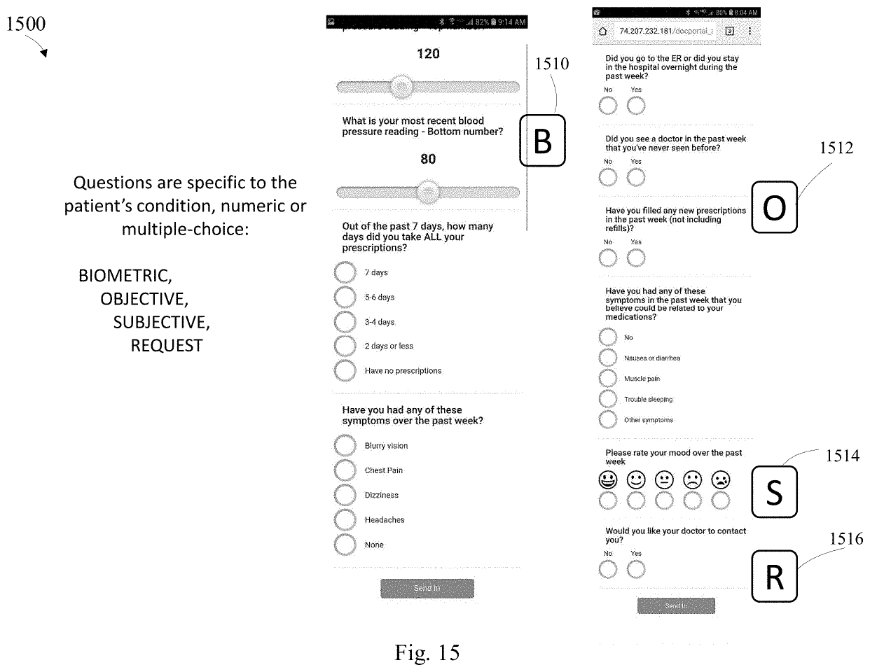

FIG. 15 depicts example questions asked of a patient, these questions fall into one of three categories biometric, objective and subjective. Biometric questions 1510 are ones that have concrete device provided data, such as blood pressure and fasting glucose levels. Since these numbers are provided by a device, they are not subject to interpretation. Objective questions 1512 are questions asked of the patient that are also fact based, but not device derived, examples of these may be “out of the last seven days, how many days did you take all of your prescriptions?”, “Have you had any of the following symptoms over the past week?”, “Did you go to the ER or did you stay in the hospital overnight during the past week?”, “Did you see a doctor over the past week that you have never seen before?” and the like. Subjective questions 1514 rely more on a patient's perceptions such as “Please rate your mood over the last week” or “Have you lost either a sense of taste or smell over the last week”. Although subjective questions do rely on a patient's perception, it may indicate a specific medical condition or reaction to a medication. The patient may also be asked “Would you like for the doctor to contact you?”. This type of request is also considered by the system when assessing a patient. If a patient requests to see a doctor, there may be the concern of the seriousness of the condition that may not be directly assessed by the model and the questions asked.

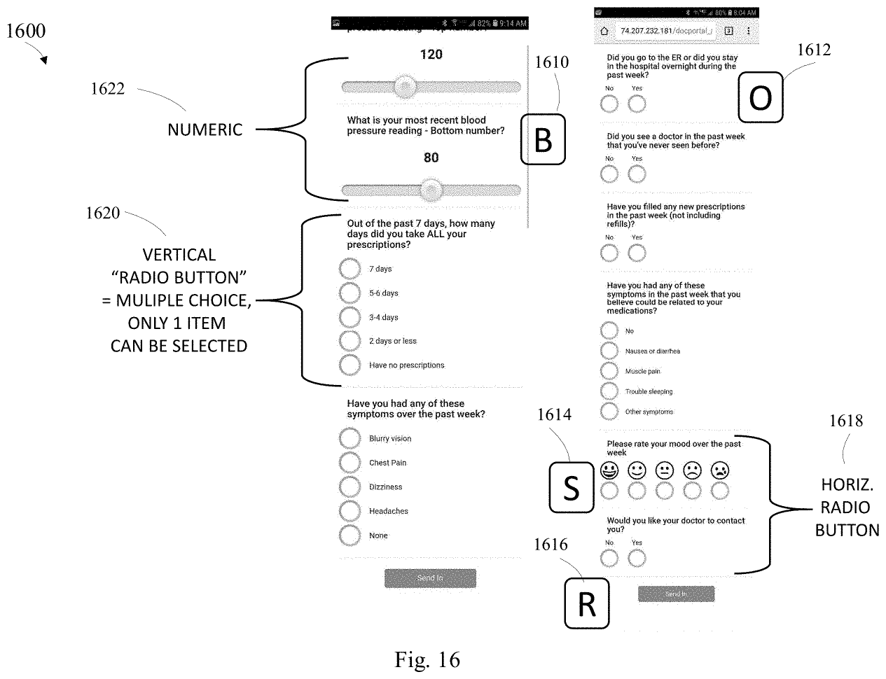

FIG. 16 depicts an example of the layout of the questions asked by the system. Biometric queries 1610 that are input numerically may use a slider 1622 to indicate the input. Objective questions 1612 put to a patient may be vertically placed radio buttons if only one answer is allowed 1620 or may be side to side radio buttons in the case of a yes or no response. Subjective questions 1614 may be indicated utilizing a horizontal radio button 1618. Requests 1616 having a yes or no response may be place side by side radio buttons.

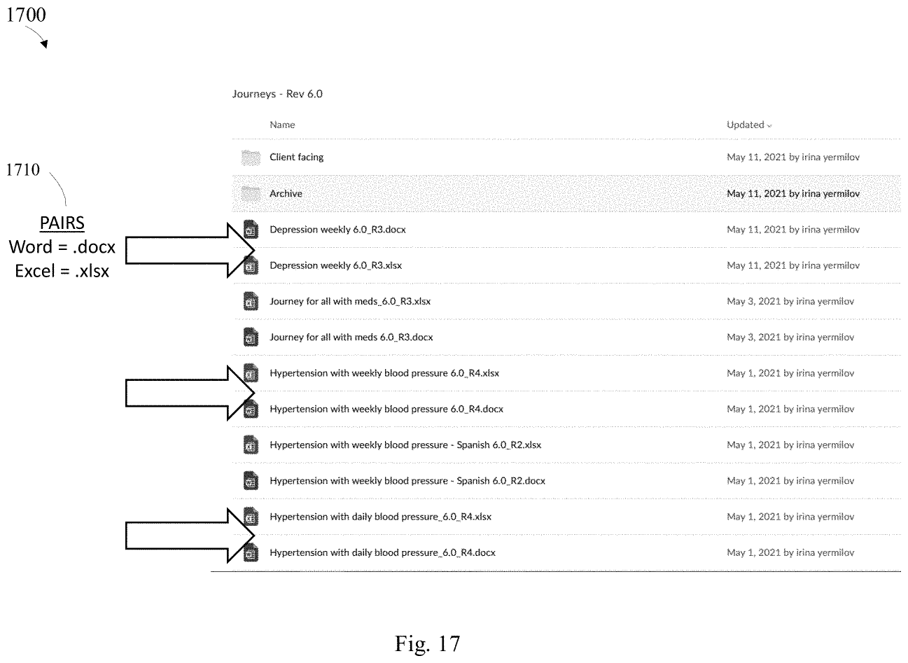

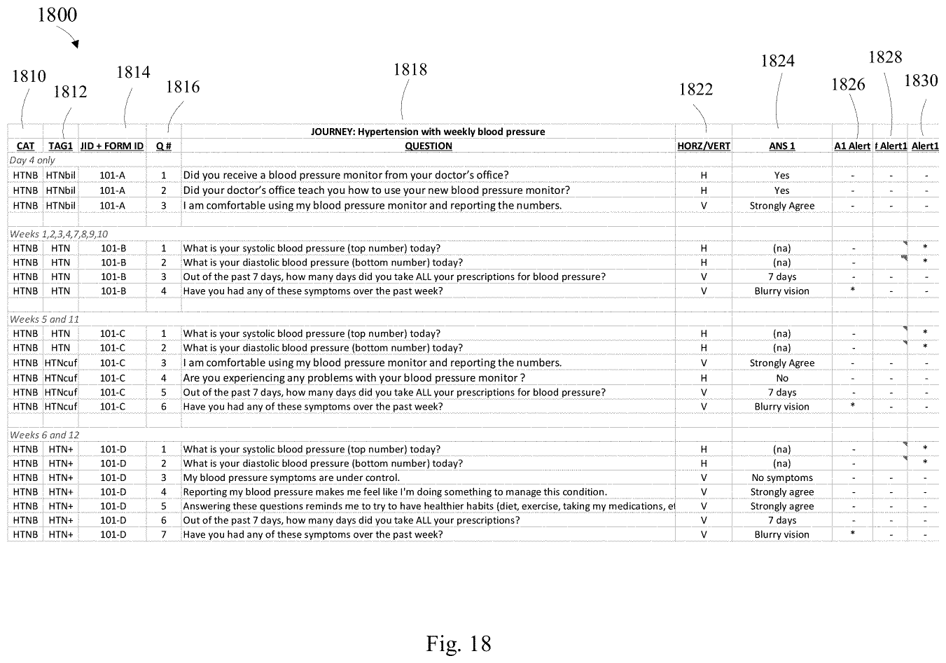

FIG. 17 depicts an example of the files used to program the system. A Word document and an Excel document are paired 1710 for each journey. The doctor will review the Word document to ensure that each of the questions asked and guardrails set are correct for their patient. The Word document information is translated into an Excel format for use by the system in setting up the specific patient journey. An example snippet from the translated Excel file is shown in FIG. 18.

FIG. 18 shows an example Excel file with some information for the hypertension and weekly blood pressure journey 1818. The questions are grouped into four sets, day 4 only, weeks 1-4 and 7-10, weeks 5 and 11 and weeks 6 and 12. The category column 1810 is indicated, the tag1 column 1812 is broken into several types for this example representing different types of questions for the different weeks. The Journey ID and Form ID (JID+FORM ID) column 1814 is grouped with the four sets discussed above day 4 only being 101-A, weeks 1-4 and 7-10 being 101-B, weeks 5 and 11 being 101-C and weeks 6 and 12 being 101-D. The questions number 1816 are shown, and the question associated with the number is shown immediately to the left of the question number for that JID+FORM ID. The direction of the radio buttons for the question is shown in the HORZ/VERT column 1822 and answer 1 is shown in column 1824 with alert trip parameters shown in columns 1826-1830.

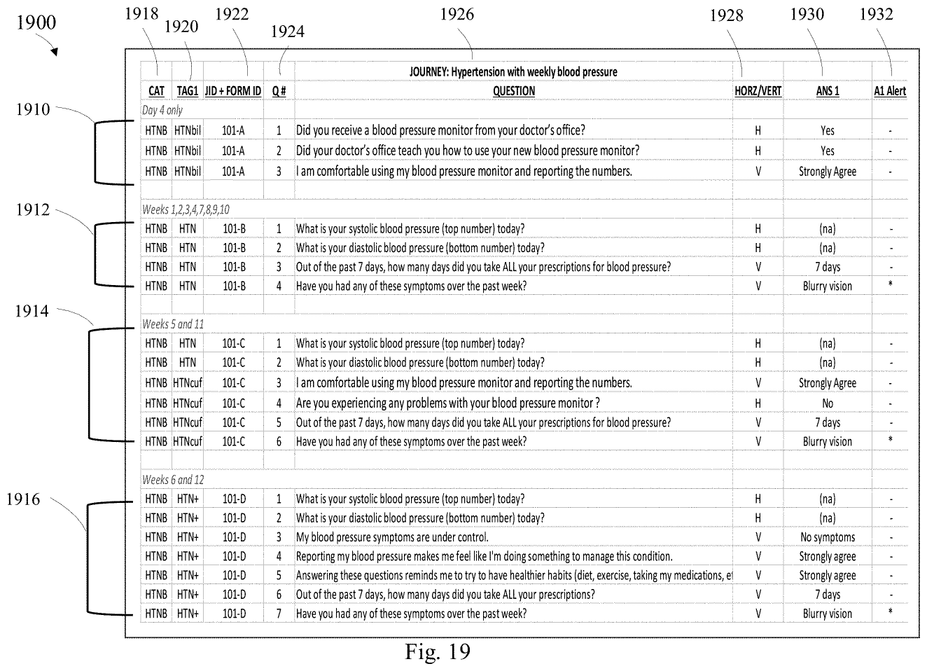

FIG. 19 shows another example Excel file with some information for the hypertension and weekly blood pressure journey 1926. The questions are grouped into four sets, day 4 only 1910, weeks 1-4 and 7-10 1912, weeks 5 and 11 1914 and weeks 6 and 12 1916. The category column 1918 is indicated, the tag1 column 1920 is broken into several types for this example representing different types of questions for the different groups 1910, 1912, 1914 and 1916. The JID+FORM ID column 1922 is grouped with the four sets discussed above 101-A for 1910, 101-B for 1912, 101-C for 1914 and 101-D for 1916. The questions number 1924 are shown, and the question associated with the number is shown immediately to the left of the question number for that JID+FORM ID. The layout of the radio buttons for the question is shown in the HORZ/VERT column 1928 and answer 1 is shown in column 1930 with alert trip points shown in column 1932.

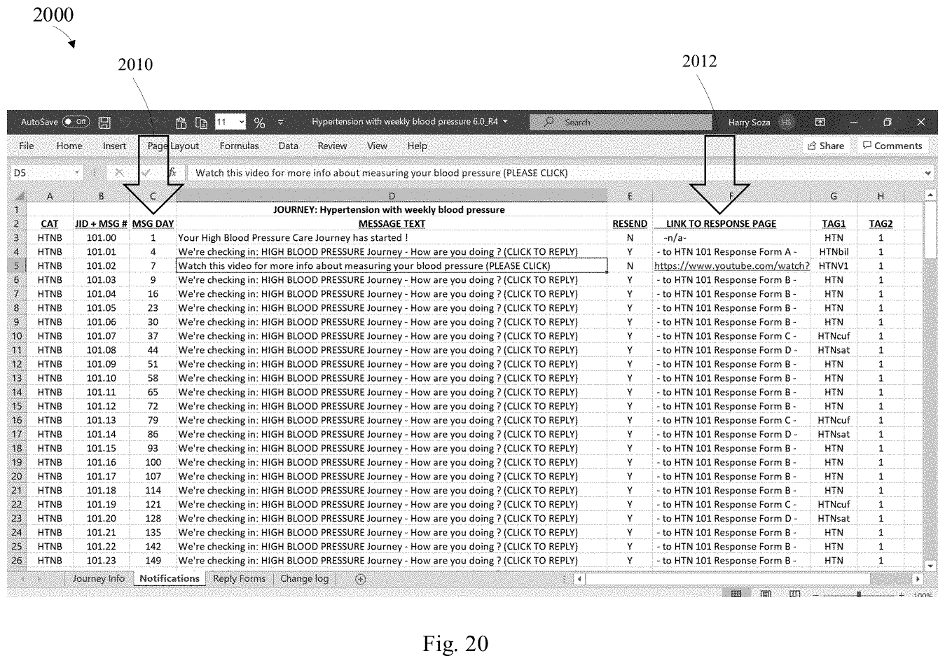

FIG. 20 shows an example of message texts associated with the hypertension and weekly blood pressure journey, specifically the texts are linked based on the category, message number and message day 2010 and a link to response page 2012.

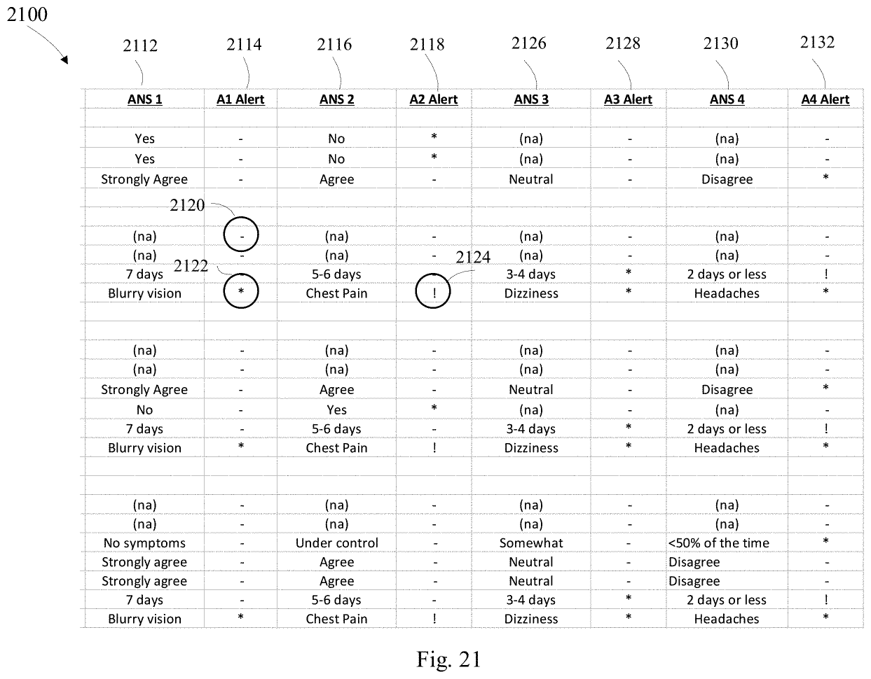

FIG. 21 depicts a portion of the Excel form and indicates answers to particular questions in 2112-2118 and 2126-2132 and if a specific answer is given the alert level, with “-” 2120 being nominal, “*” 2122 indicating warning and “!” 2124 indicating critical.

FIG. 22 depicts an example of guardrail values and their possible resetting by medical personnel. In this example the alert levels include “-” being nominal, “*” indicating warning and “!” indicating critical. The guardrails 2210 for systolic blood pressure are shown as 2212 for low blood pressure emergency, 2214 for low critical blood pressure, 2216 for low blood pressure warning, 2218 for high blood pressure warning, 2220 for high critical blood pressure and 2222 for high emergency blood pressure. These are independently set for the systolic and diastolic blood pressures. The blood pressure alert level guardrails may be modified by the medical personnel and the dates for the modifications and the levels indicated in 2224-2228 to prevent false alarms.

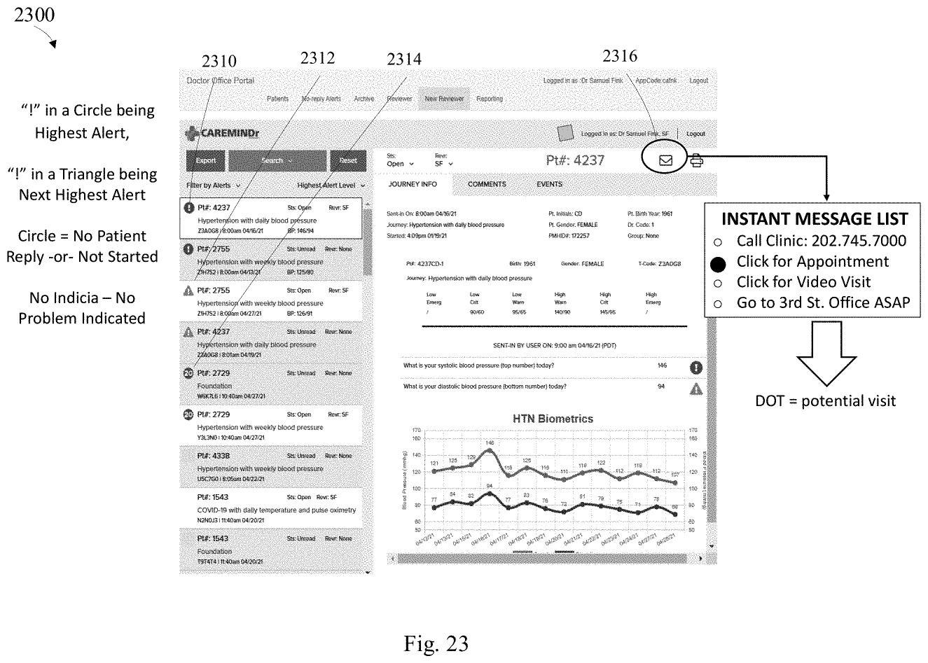

FIG. 23 depicts a review chart having the most critical patients sorted by criticality. In this chart a “!” within a circle is of highest criticality 2310 and a “!” within a triangle 2312 is of next highest criticality. A circle 2314 without an exclamation point is an item to be reviewed by medical personnel and responses without indicia indicate that no problem was detected. In this example the highest criticality case 2310, is for patient PT #:4237, a biometric history of the blood pressure values is shown in the bottom right hand side of the screen and at the top right is a letter symbol 2316 that allows the medical personnel to send a message that may either ask the patient to call the clinic, click for an appointment, ask the patient to watch a video or immediately go to a medical office.

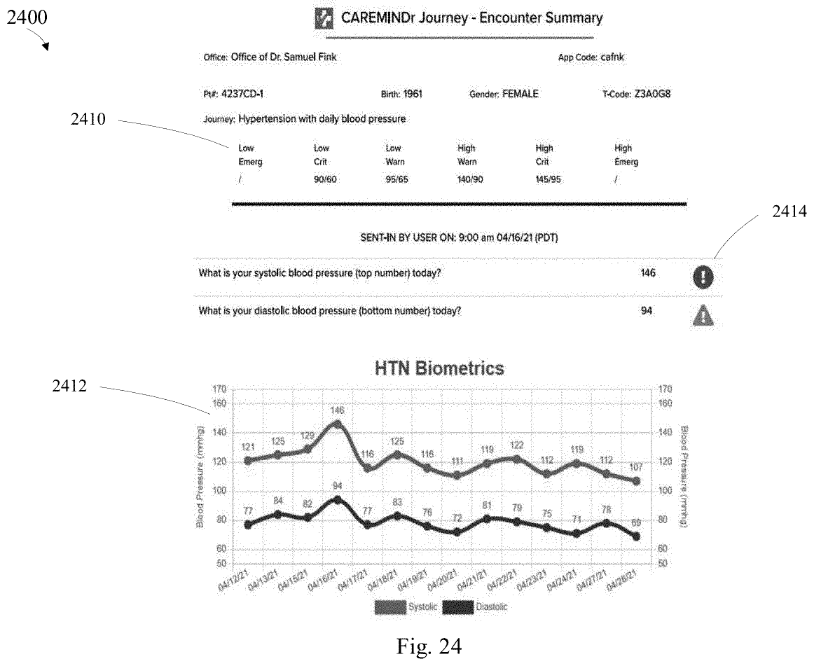

FIG. 24 is a review of the topmost critical patient from FIG. 23, patient PT #:4237. In this chart the guardrails are indicated 2410, the biometric history is shown in 2412 and the specific alert is shown in 2414.

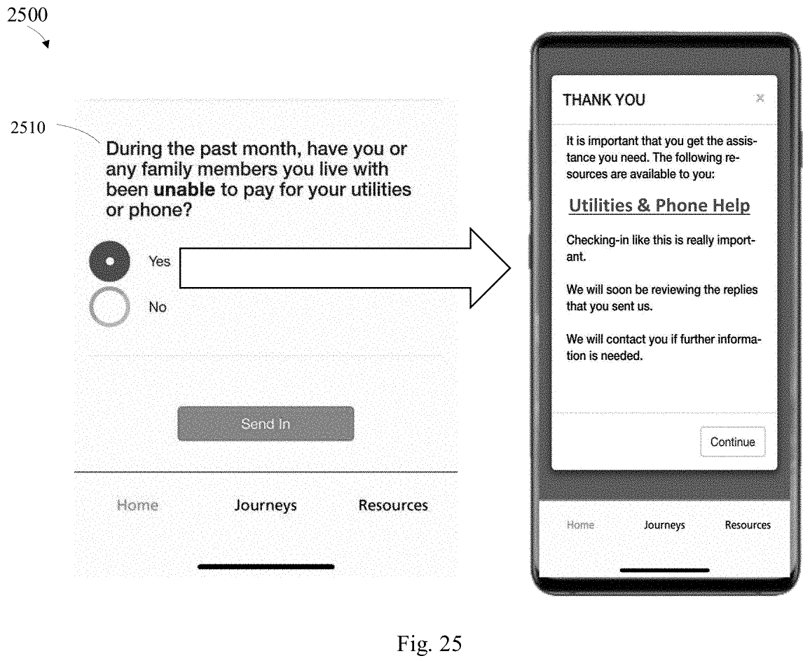

FIG. 25 indicates environmental condition questions that may have an impact on health issues. In this instance the question is asked whether the patient during the last month has been unable to pay utility bills or phone bills 2510. The system then may send the patient a link for help in paying the utility or phone bills. The inability to afford to pay for services, see the doctor or get to the medical office may affect a patient's health.

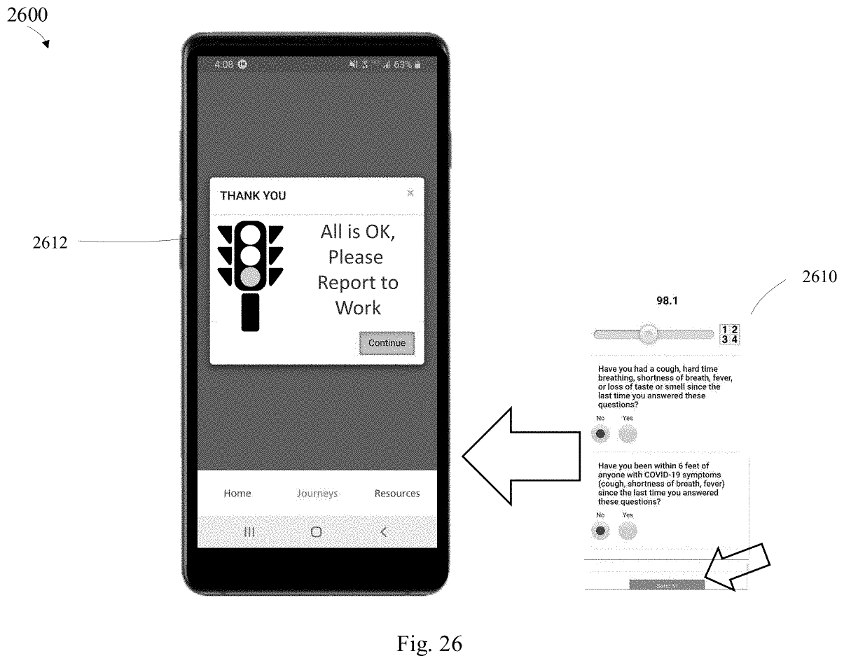

FIG. 26 indicates example questions related to spreading illnesses such as COVID-19 to fellow workers at a workplace. The system asks for the temperature of the worker 2610 and asks the questions pertaining to cough, hard time breathing or loss of taste or smell and whether they have been in contact with someone who has COVID-19. If the answers to the questions indicate a low risk having the disease the worker is instructed that it is okay to return to work 2612.

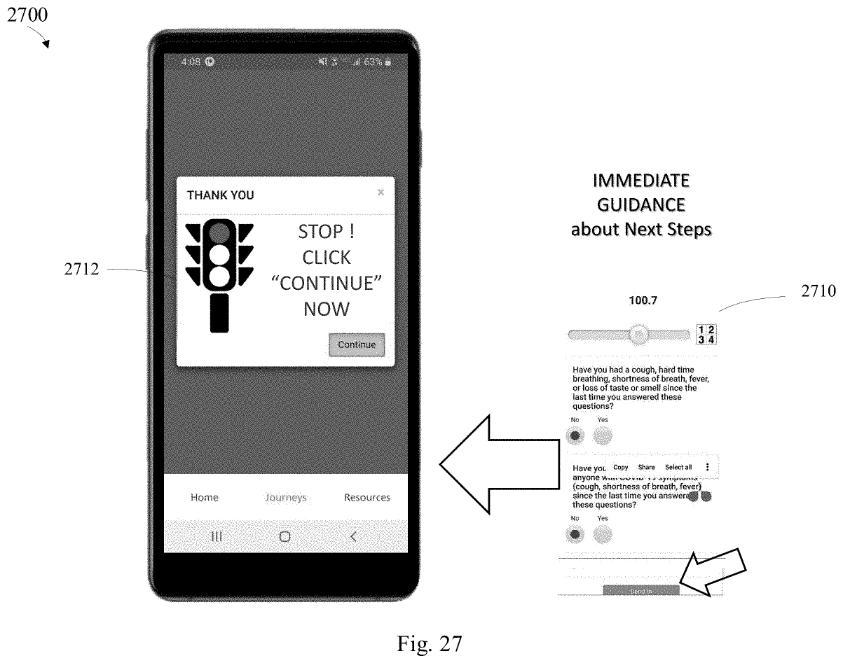

FIG. 27 also indicates example questions related to spreading illnesses such as COVID-19 to fellow workers at a workplace. In this instance the patient has a slight fever of 100.7 2710 and is instructed to stop and press continue 2712 in which case protocols for handling a possible case of COVID-19 are followed.

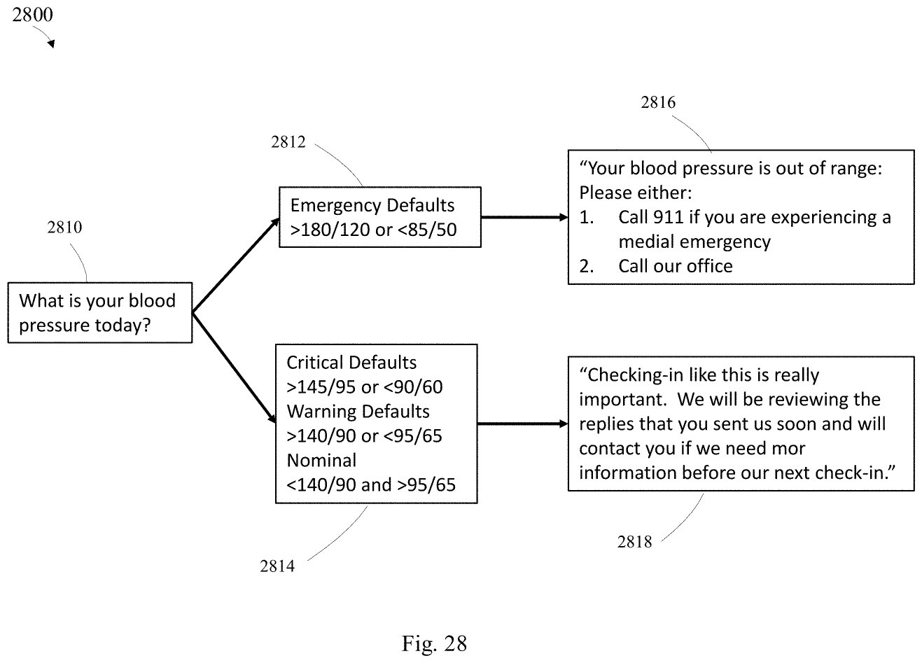

FIG. 28 depicts an example of a hypertension and weekly blood pressure journey message flow for different blood pressures. In this example the patient is asked what their blood pressure level was today 2810. An emergency would be indicated by levels above 180/120 or less than 85/50 2812. The response to the emergency would be to either call 911 or immediately call the doctors office 2816. If the blood pressure levels are not an emergency, it may be either nominal at less than 140/90 or greater than 95/65, a warning at greater than 140/90 or less than 95/65 or critical at greater than 145/95 or less than 90/60 2814. The system would send a response that the data is being reviewed and that the office may contact the patient if more information is needed 2818.

The operations of a method or algorithm described in connection with the embodiments disclosed herein may be embodied directly in hardware, in a computer program executed by a processor, or in a combination of the two. A computer program may be embodied on a computer readable medium, such as a storage medium. For example, a computer program may reside in random access memory (“RAM”), flash memory, read-only memory (“ROM”), erasable programmable read-only memory (“EPROM”), electrically erasable programmable read-only memory (“EEPROM”), registers, hard disk, a removable disk, a compact disk read-only memory (“CD-ROM”), or any other form of storage medium known in the art.

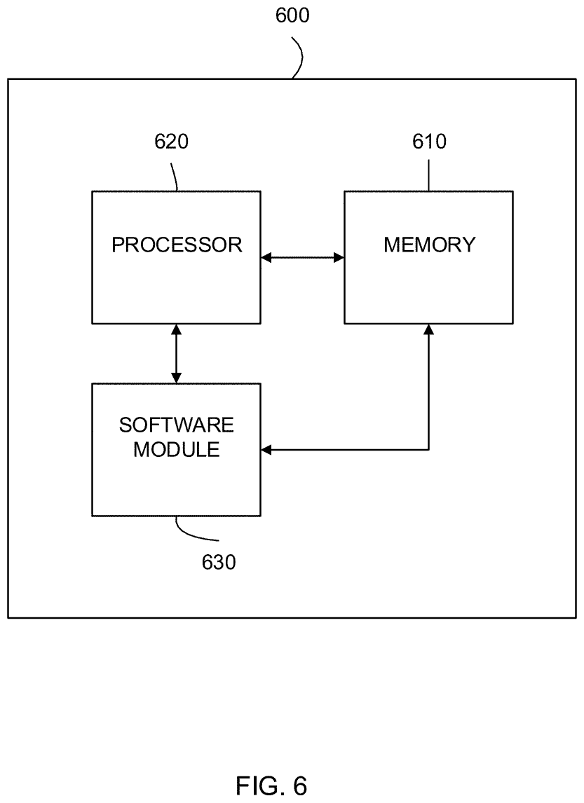

An exemplary storage medium may be coupled to the processor such that the processor may read information from, and write information to, the storage medium. In the alternative, the storage medium may be integral to the processor. The processor and the storage medium may reside in an application specific integrated circuit (“ASIC”). In the alternative, the processor and the storage medium may reside as discrete components. For example, FIG. 6 illustrates an example network element, which may represent any of the above-described network components of the other figures.

As illustrated in FIG. 6, a memory 610 and a processor 620 may be discrete components of the network entity 600 that are used to execute an application or set of operations. The application may be coded in software in a computer language understood by the processor 620, and stored in a computer readable medium, such as, the memory 610. The computer readable medium may be a non-transitory computer readable medium that includes tangible hardware components in addition to software stored in memory. Furthermore, a software module 630 may be another discrete entity that is part of the network entity 600, and which contains software instructions that may be executed by the processor 620. In addition to the above noted components of the network entity 600, the network entity 600 may also have a transmitter and receiver pair configured to receive and transmit communication signals (not shown).

Although an exemplary embodiment of the system, method, and computer readable medium of the present application has been illustrated in the accompanied drawings and described in the foregoing detailed description, it will be understood that the application is not limited to the embodiments disclosed, but is capable of numerous rearrangements, modifications, and substitutions without departing from the spirit or scope of the application as set forth and defined by the following claims. For example, the capabilities of the system of the various figures can be performed by one or more of the modules or components described herein or in a distributed architecture and may include a transmitter, receiver or pair of both. For example, all or part of the functionality performed by the individual modules, may be performed by one or more of these modules. Further, the functionality described herein may be performed at various times and in relation to various events, internal or external to the modules or components. Also, the information sent between various modules can be sent between the modules via at least one of: a data network, the Internet, a voice network, an Internet Protocol network, a wireless device, a wired device and/or via plurality of protocols. Also, the messages sent or received by any of the modules may be sent or received directly and/or via one or more of the other modules.

One skilled in the art will appreciate that a “system” could be embodied as a personal computer, a server, a console, a personal digital assistant (PDA), a cell phone, a tablet computing device, a smartphone or any other suitable computing device, or combination of devices. Presenting the above-described functions as being performed by a “system” is not intended to limit the scope of the present application in any way, but is intended to provide one example of many embodiments of the present application. Indeed, methods, systems and apparatuses disclosed herein may be implemented in localized and distributed forms consistent with computing technology.

It should be noted that some of the system features described in this specification have been presented as modules, in order to more particularly emphasize their implementation independence. For example, a module may be implemented as a hardware circuit comprising custom very large-scale integration (VLSI) circuits or gate arrays, off-the-shelf semiconductors such as logic chips, transistors, or other discrete components. A module may also be implemented in programmable hardware devices such as field programmable gate arrays, programmable array logic, programmable logic devices, graphics processing units, or the like.

A module may also be at least partially implemented in software for execution by various types of processors. An identified unit of executable code may, for instance, comprise one or more physical or logical blocks of computer instructions that may, for instance, be organized as an object, procedure, or function. Nevertheless, the executables of an identified module need not be physically located together but may comprise disparate instructions stored in different locations which, when joined logically together, comprise the module and achieve the stated purpose for the module. Further, modules may be stored on a computer-readable medium, which may be, for instance, a hard disk drive, flash device, random access memory (RAM), tape, or any other such medium used to store data.

Indeed, a module of executable code could be a single instruction, or many instructions, and may even be distributed over several different code segments, among different programs, and across several memory devices. Similarly, operational data may be identified and illustrated herein within modules and may be embodied in any suitable form and organized within any suitable type of data structure. The operational data may be collected as a single data set or may be distributed over different locations including over different storage devices, and may exist, at least partially, merely as electronic signals on a system or network.

It will be readily understood that the components of the application, as generally described and illustrated in the figures herein, may be arranged and designed in a wide variety of different configurations. Thus, the detailed description of the embodiments is not intended to limit the scope of the application as claimed but is merely representative of selected embodiments of the application.

One having ordinary skill in the art will readily understand that the application as discussed above may be practiced with steps in a different order, and/or with hardware elements in configurations that are different than those which are disclosed. Therefore, although the application has been described based upon these preferred embodiments, it would be apparent to those of skill in the art that certain modifications, variations, and alternative constructions would be apparent, while remaining within the spirit and scope of the application. In order to determine the metes and bounds of the application, therefore, reference should be made to the appended claims.

While preferred embodiments of the present application have been described, it is to be understood that the embodiments described are illustrative only and the scope of the application is to be defined solely by the appended claims when considered with a full range of equivalents and modifications (e.g., protocols, hardware devices, software platforms etc.) thereto.

What is claimed is:

1. A method, comprising:
linking a mobile device and a health care provider server;
requesting a patient response to a message including a query of a health-related issue, wherein the response is at least one of biometric, objective and subjective;
receiving the patient response to the query;
determining based on at least one predetermined alert criteria an urgency level of the patient response based on the health-related issue;
tagging the patient response as an urgent tagged response if the determined urgency level exceeds a predetermined urgency threshold of the patient for the health-related issue based on the at least one predetermined alert criteria; and
providing the request and the urgent tagged response to a healthcare provider.

2. The method of claim 1 further comprising sending at least one follow-on query based on the urgent tagged response.

3. The method of claim 1 further comprising grading the urgency level of the urgent tagged response, wherein the grading is at least one of an immediate medical action advisory, a follow-up advisory and a medical history review advisory.

4. The method of claim 1 further comprising tagging the response as a non-urgent tagged response if the determined urgency level does not meet the predetermined urgency threshold of the patient for the health-related issue.

5. The method of claim 4 further comprising proposing a set of workflow instructions linked to at least one of the urgent tagged response and the non-urgent tagged response.

6. The method of claim 1 further comprising proposing a set of workflow instructions linked to the urgent tagged response.

7. The method of claim 6 further comprising presenting a clinical escalation review advisory linked to the urgent tagged response.

8. The method of claim 1 wherein the request is provided to the patient at a clinically relevant time interval based on the health-related issue.

9. The method of claim 1, further comprising:
selecting a treatment plan for a patient comprising a set of treatment information;
linking an application identifier and a T-code identifier to the treatment plan;
launching a treatment plan application;
retrieving the set of treatment information;
populating the treatment plan application with the set of treatment information; and
triggering a message dispatch in accordance with the treatment plan, the message dispatch including the query to the health-related issue to determine a patient status.

10. A non-transitory computer readable medium comprising instructions that, when read by a processor, cause the processor to perform:
linking a mobile device and a health care provider server;
requesting from a patient pertaining to a health-related issue a response to a query, wherein the response is at least one of biometric, objective and subjective;
receiving the response to the query;
determining based on at least one predetermined alert criteria an urgency level of the response based on the patient health-related issue;
tagging the response as an urgent tagged response if the determined urgency level exceeds a predetermined urgency threshold of the patient for the health-related issue based on the at least one predetermined alert criteria; and
providing the request and the urgent tagged response a healthcare provider.

11. The non-transitory computer readable medium of claim 10, further comprising:
selecting a treatment plan for the patient comprising a set of treatment information;
linking an application identifier and a T-code identifier to the treatment plan;
launching a treatment plan application;
retrieving the set of treatment information;
populating the treatment plan application with the set of treatment information; and
triggering a message dispatch in accordance with the treatment plan, the message dispatch including the query to the health-related issue to determine a patient status.

12. The non-transitory computer readable medium of claim 11, further comprising:
loading a trigger table having a set of trigger dates based on the treatment plan, the message dispatch responsive to the set of trigger dates; and
receiving a message start date and receiving an initialization message from a patient mobile device to initiate the treatment plan and to initialize the set of trigger dates in the trigger table.

13. The non-transitory computer readable medium of claim 11, further comprising at least one of creating the treatment plan and modifying the treatment plan.

14. The non-transitory computer readable medium of claim 10, further comprising sending at least one follow-on query based on the urgent tagged response.

15. The non-transitory computer readable medium of claim 10, further comprising grading the urgency level of the urgent tagged response, wherein the grading is at least one of an immediate medical action advisory, a follow-up advisory and a medical history review advisory.

16. The non-transitory computer readable medium of claim 10, further comprising:
tagging the response as a non-urgent tagged response if the determined urgency level does not meet the predetermined urgency threshold of the patient for the health-related issue; and
proposing a set of workflow instructions linked to at least one of the urgent tagged response and the non-urgent tagged response.

17. The non-transitory computer readable medium of claim 10, further comprising:
proposing a set of workflow instructions linked to the urgent tagged response; and
presenting a clinical escalation review advisory linked to the urgent tagged response.

18. A system, comprising:
at least one cloud-based processor; and
at least one memory electrically coupled to the at least one processor and storing an application, wherein the processor performs operations to:
link a mobile device and a health care provider server;
request from a patient pertaining to a health-related issue a response to a query;
receive the response to the query;
determine an urgency level of the response based on the patient health-related issue;
tag the response as an urgent tagged response if the determined urgency level exceeds a predetermined urgency threshold of the patient for the health-related issue; and
provide the request and the urgent tagged response a healthcare provider.

19. The system of claim 18, wherein the processor further performs an operation to:
tag the response as a non-urgent tagged response if the determined urgency level does not meet the predetermined urgency threshold of the patient for the health-related issue; and
propose a set of workflow instructions linked to at least one of the urgent tagged response and the non-urgent tagged response.

20. The system of claim 18, wherein the processor further performs an operation to send at least one follow-on query based on the urgent tagged response.
